SEQUENCE LISTING (53 sequences)

SEQ ID NO: 1 - protein, 32 aa, Cicer arietinum
TKPCQSDKDCKKFACRKPKVPKCINGFCKCVR

SEQ ID NO: 2 - protein, 32 aa, synthetic construct
  REGION 1..32: note = synthetic
TKPCQSDKDCKWFACRKPKWPKCINGFCKCVR

SEQ ID NO: 3 - protein, 32 aa, synthetic construct
  REGION 1..32: note = synthetic
TKPCQSIKICKKFACRKPKVPKCINGFCKCVR

SEQ ID NO: 4 - protein, 32 aa, synthetic construct
  REGION 1..32: note = synthetic
TKPCQSSKPCKWFACRKPKWPKCINGFCKCVR

SEQ ID NO: 5 - protein, 32 aa, synthetic construct
  REGION 1..32: note = synthetic
TKPCQSSKPCKWFACRKPKWPKCINGFCFCVR

SEQ ID NO: 6 - protein, 7 aa, synthetic construct
  REGION 1..7: note = synthetic
FACRKPK

SEQ ID NO: 7 - protein, 10 aa, synthetic construct
  REGION 1..10: note = synthetic
KWFACRKPKW

SEQ ID NO: 8 - protein, 34 aa, Cicer arietinum
KKMPCKRRRDCKTYPCPHPKVRDCVKGYCKCVVR

SEQ ID NO: 9 - protein, 34 aa, synthetic construct
  REGION 1..34: note = synthetic
KKMPCKRRRDCKWYPCPHPKWRDCVKGYCKCVVR

SEQ ID NO: 10 - protein, 34 aa, synthetic construct
  REGION 1..34: note = synthetic
KKMPCKRRRICKTYPCPHPKVRDCVKGYCKCVVR

SEQ ID NO: 11 - protein, 34 aa, synthetic construct
  REGION 1..34: note = synthetic
KKMPCKRRRPCKWYPCPHPKWRDCVKGYCKCVVR

SEQ ID NO: 12 - protein, 34 aa, synthetic construct
  REGION 1..34: note = synthetic
KKMPCKRRRPCKWYPCPHPKWRDCVKGYCFCVVR

SEQ ID NO: 13 - protein, 7 aa, synthetic construct
  REGION 1..7: note = synthetic
YPCPHPK

SEQ ID NO: 14 - protein, 10 aa, synthetic construct
  REGION 1..10: note = synthetic
KWYPCPPSKW

SEQ ID NO: 15 - protein, 26 aa, Cicer arietinum
SRDCKTYPCPPSKVKDCIKGYCKCVR

SEQ ID NO: 16 - protein, 26 aa, synthetic construct
  REGION 1..26: note = synthetic
SRDCKWYPCPPSKWKDCIKGYCKCVR

SEQ ID NO: 17 - protein, 26 aa, synthetic construct
  REGION 1..26: note = synthetic
SRICKWYPCPPSKWKDCIKGYCKCVR

SEQ ID NO: 18 - protein, 26 aa, synthetic construct
  REGION 1..26: note = synthetic
SRPCKWYPCPPSKWKDCIKGYCKCVR

SEQ ID NO: 19 - protein, 26 aa, synthetic construct
  REGION 1..26: note = synthetic
SRPCKWYPCPPSKWKDCIKGYCFCVR

SEQ ID NO: 20 - protein, 7 aa, synthetic construct
  REGION 1..7: note = synthetic
YPCPPSK

SEQ ID NO: 21 - protein, 10 aa, synthetic construct
  REGION 1..10: note = synthetic
KWYPCPPSKW

SEQ ID NO: 22 - protein, 33 aa, Cicer arietinum
TKQPCKSRKHCKTYRCPTPKVPNCVNGFCKCVR

SEQ ID NO: 23 - protein, 33 aa, synthetic construct
  REGION 1..33: note = synthetic
TKQPCKSRKHCKWYRCPTPKWPNCVNGFCKCVR

SEQ ID NO: 24 - protein, 33 aa, synthetic construct
  REGION 1..33: note = synthetic
TKQPCKSRKHCKWYRCPTPKWPNCVNGFCFCVR

SEQ ID NO: 25 - protein, 31 aa, synthetic construct
  REGION 1..31: note = synthetic
QPCKSRKHCKWYRCPTPKWPNCVNGFCKCVR

SEQ ID NO: 26 - protein, 31 aa, synthetic construct
  REGION 1..31: note = synthetic
QPCKSRKHCKWYRCPTPKWPNCVNGFCFCVR

SEQ ID NO: 27 - protein, 7 aa, synthetic construct
  REGION 1..7: note = synthetic
YRCPTPK

SEQ ID NO: 28 - protein, 10 aa, synthetic construct
  REGION 1..10: note = synthetic
KWYRCPTPKW

SEQ ID NO: 29 - skipped

SEQ ID NO: 30 - protein, 10 aa, synthetic construct
  REGION 1..10: note = synthetic
  VARIANT 3: note = Xaa is phenylalanine or tyrosine
  VARIANT 4: note = Xaa alanine, tryptophan, phenylalanine, arginine, or proline
  VARIANT 6: note = Xaa arginine or proline
  VARIANT 7: note = Xaa is lysine, histidine,threonine, or proline
  VARIANT 8: note = Xaa is proline or serine
KWXXCXXXKW

SEQ ID NO: 31 - protein, 4 aa, synthetic construct
  REGION 1..4: note = synthetic
GGGG

SEQ ID NO: 32 - protein, 5 aa, synthetic construct
  REGION 1..5: note = synthetic
GGGGS

SEQ ID NO: 33 - protein, 6 aa, synthetic construct
  REGION 1..6: note = synthetic
SGGGGS

SEQ ID NO: 34 - protein, 32 aa, synthetic construct
  REGION 1..32: note = synthetic
TKPCQSAKACKWFWCRKPKWPKCINGFCKCVR

SEQ ID NO: 35 - protein, 32 aa, synthetic construct
  REGION 1..32: note = synthetic
TKPCQSLKLCKKFWCRKPKWPKCINGFCKCVR

SEQ ID NO: 36 - protein, 32 aa, synthetic construct
  REGION 1..32: note = synthetic
TKPCQSVKVCKWFWCRKPKWPKCINGFCKCVR

SEQ ID NO: 37 - protein, 32 aa, synthetic construct
  REGION 1..32: note = synthetic
TKPCQSIKICKWFWCRKPKWPKCINGFCFCVR

SEQ ID NO: 38 - protein, 10 aa, synthetic construct
  REGION 1..10: note = synthetic
KWFWCRKPKW

SEQ ID NO: 39 - protein, 10 aa, synthetic construct
  REGION 1..10: note = synthetic
KWYPCPPSKW

SEQ ID NO: 40 - protein, 10 aa, synthetic construct
  REGION 1..10: note = synthetic
KWYWCPPSKW

SEQ ID NO: 41 - protein, 32 aa, synthetic construct
  REGION 1..32: note = synthetic
TKPCAAAAACKKFACRKPKVPKCINGFCKCVR

SEQ ID NO: 42 - protein, 32 aa, synthetic construct
  REGION 1..32: note = synthetic
TKPCQSDKDCAAAACRKPKVPKCINGFCKCVR

SEQ ID NO: 43 - protein, 32 aa, synthetic construct
  REGION 1..32: note = synthetic
TKPCQSDKDCKKFACAAAAVPKCINGFCKCVR

SEQ ID NO: 44 - protein, 32 aa, synthetic construct
  REGION 1..32: note = synthetic
TKPCQSDKDCKKFACRKPKAAACINGFCKCVR

SEQ ID NO: 45 - protein, 32 aa, synthetic construct
  REGION 1..32: note = synthetic
TKPCQSDKDCKKFACRKPKVPKCAAAACKCVR

SEQ ID NO: 46 - protein, 32 aa, synthetic construct
  REGION 1..32: note = synthetic
TKPCQSAKACKKFWCRKPKWPKCINGFCKCVR

SEQ ID NO: 47 - protein, 32 aa, synthetic construct
  REGION 1..32: note = synthetic
TKPCQSAKACKKFWCRKPKWPKCINGFCFCVR

SEQ ID NO: 48 - protein, 32 aa, synthetic construct; identical to SEQ ID NO: 35
  REGION 1..32: note = synthetic

SEQ ID NO: 49 - protein, 32 aa, synthetic construct
  REGION 1..32: note = synthetic
TKPCQSLKLCKKFWCRKPKWPKCINGFCFCVR

SEQ ID NO: 50 - protein, 32 aa, synthetic construct
  REGION 1..32: note = synthetic
TKPCQSVKVCKKFWCRKPKWPKCINGFCKCVR

SEQ ID NO: 51 - protein, 32 aa, synthetic construct
  REGION 1..32: note = synthetic
TKPCQSVKVCKKFWCRKPKWPKCINGFCFCVR

SEQ ID NO: 52 - protein, 32 aa, synthetic construct
  REGION 1..32: note = synthetic
TKPCQSIKICKKFWCRKPKWPKCINGFCKCVR

SEQ ID NO: 53 - protein, 32 aa, synthetic construct
  REGION 1..32: note = synthetic
TKPCQSIKICKKFWCRKPKWPKCINGFCFCVR

(19) United States
(12) Patent Application Publication
(10) Pub. No.: US 2025/0176546 A1
(43) Pub. Date: Jun. 5, 2025

(54) ANTIMICROBIAL CaNCR PEPTIDE VARIANTS

(71) Applicant: Donald Danforth Plant Science Center, ST. LOUIS, MO (US)

(72) Inventors: Dilip M. Shah, Chesterfield, MO (US); Meenakshi Tetorya, St. Louis, MO (US)

(21) Appl. No.: 18/862,046

(22) PCT Filed: May 2, 2023
(86) PCT No.: PCT/US2023/066509

§ 371 Date: Oct. 31, 2024

Related U.S. Application Data
(60) Provisional application No. 63/364,076, filed on May 3, 2022.

(51) Int. Cl. A01N 37/46; A01P 1/00; A61K 38/00; A61P 31/10; C07K 14/47

(52) U.S. Cl. CPC A01N 37/46; A01P 1/00; A61P 31/10; C07K 14/4723; A61K 38/00

(57) ABSTRACT

Antimicrobial CaNCR peptide variants or CaNCR peptide variant multimers are disclosed along with compositions comprising the CaNCR peptide variants or CaNCR peptide variant multimers and transgenic or genetically edited plants or microorganisms that express the CaNCR peptide variants or CaNCR peptide variant multimers to inhibit growth of pathogenic microbes. Such CaNCR peptide variants, or CaNCR peptide variant multimers, compositions, plants, and microorganisms can provide for inhibition of microbial growth in plants, processed plant products, animals, and humans.

### CROSS-REFERENCE TO RELATED APPLICATIONS

The present application claims priority to and the benefit of U.S. Provisional Application No. 63/364,076, filed May 3, 2022, the contents of which are herein incorporated by reference in their entirety.

### STATEMENT REGARDING FEDERALLY SPONSORED RESEARCH

This invention was made with government support under National Science Foundation Award Number 2037981 awarded by the National Science Foundation. The government has certain rights in the invention.

### REFERENCE TO SEQUENCE LISTING SUBMITTED ELECTRONICALLY

The present application includes an XML sequence listing document entitled “225312-528100_Sequence-listing” with a file size of 66,628 bytes (measured in MS-Windows), which was created on Apr. 28, 2023 and is incorporated herein by reference in its entirety. The XML sequence listing document was converted from the ST.25 sequence listing text document filed with the priority application 63/364,076 on May 3, 2022.

### FIELD

The present disclosure relates to CaNCR peptide variants and recombinant or edited polynucleotides encoding the CaNCR peptide variants. The antimicrobial CaNCR peptide variants can be applied directly to a plant, human, or animal, applied to a plant in the form of microorganisms that produce the peptides, or the plants can be genetically transformed or edited to produce the peptides or proteins. The present disclosure also relates to recombinant polynucleotides, edited polynucleotides, edited genomes, microorganisms and plants comprising those polynucleotides or genomes, and compositions useful in controlling pathogenic microbes.

### BACKGROUND

Protection of agriculturally important crops from pathogenic microbes (e.g., fungi, including mold, yeast and dimorphic fungi, or oomycetes) is crucial in improving crop yields. Fungal infections are a particular problem in damp climates and can become a major concern during crop storage, where such infections can result in spoilage and contamination of food or feed products with fungal toxins. Unfortunately, modern growing methods, harvesting and storage systems can promote plant pathogen infections.

Certain microbes (e.g., fungi, including yeast, or oomycetes) can also be pathogenic to various vertebrates including humans, fish, and the like. Control of plant pathogens is further complicated by the need to simultaneously control multiple microbes of distinct genera. For example, microbes such as Alternaria; Ascochyta; Botrytis; Cercospora; Colletotrichum; Diplodia; Erysiphe; Fusarium; Gaeumanomyces; Helminthosporium; Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Sclerotium; Sclerotinia; Septoria; Thielaviopsis; Uncinula; Venturia; and Verticillium species are all recognized plant pathogens. Consequently, resistant crop plant varieties or antimicrobial agents that control only a limited subset of microbial pathogens can fail to deliver adequate protection under conditions where multiple pathogens are present. It is further anticipated that plant pathogenic microbes can become resistant to existing antimicrobial agents and crop varieties, which can favor the introduction of new microbial control agents with distinct modes of action to combat the resistant microbes.

A group of peptides known as defensins have been shown to inhibit plant pathogens. Defensins have been previously identified as small cysteine-rich peptides of about 45-54 amino acids that constitute an important component of the innate immunity of plants (Thomma et al., 2002; Lay and Anderson, 2005; Vriens et al., 2014). Widely distributed in plants, defensins vary greatly in their amino acid composition. However, they all have a compact shape which is stabilized by either four or five intramolecular disulfide bonds. Plant defensins have been characterized as comprising a conserved γ-core motif comprising a conserved GXCX3-9C (where X is any amino acid) sequence (Lacerda et al., 2014). The three-dimensional structure of the previously characterized γ-core motif consists of two antiparallel β-sheets, with an interpolated turn region (Ibid.). Antimicrobial activity of certain defensins has been correlated with the presence of positively charged amino acid residues in the γ-core motif (Spelbrink et al., Plant Physiol., 2004; Sagaram et al., 2013).

Several publications have disclosed expression vectors that encode proteins having at least two defensin peptides that are liked by a peptide sequence that can be cleaved by plant endoproteinases (WO2014078900; Vasivarama and Kirti, 2013a; François et al.; Vasivarama and Kirti, 2013b). A MtDef5 proprotein comprising two defensin peptides separated by a small peptide linker has also been disclosed in US Patent Appl. Pub. No. 20160208278. Other multimeric defensin proteins have been disclosed in WO2017156457 and WO2017127558.

Certain nodule specific cysteine rich (NCR) peptides with antimicrobial activity expressed in nodules of Medicago truncatula (Barrel Medic) have been described (WO2010146067). Other NCR peptides from Cicer arietinum (Chickpea) have been described and implicated in the terminal differentiation of endosymbiotic bacteria (Montiel et al., 2016, Molec. Plant Microb. Inter. 29:210-219). International Patent Application publication WO2020/146360 describes antimicrobial NCR polypeptides including the CaNCR7, CaNCR13, CaNCR14, and CaNCR15 peptides as well as certain variants thereof.

### SUMMARY

CaNCR peptide variant comprising one or more amino acid substitutions in a CaNCR peptide, wherein the CaNCR peptide comprises a CaNCR13 peptide of SEQ ID NO: 1, a CaNCR7 peptide of SEQ ID NO: 8, a CaNCR14 peptide of SEQ ID NO: 15, or a CaNCR15 peptide of SEQ ID NO: 22, wherein the amino acid substitutions in the CaNCR peptide correspond to a D7I substitution, a D7S substitution, a D7A substitution, D7V substitution, D7L substitution, a D9I substitution, a D9P substitution, a D9A substitution, D9V substitution, a D9L substitution, a K12W substitution, a K29F substitution, or any combination of said substitutions in a full length reference peptide of SEQ ID NO: 1 when the CaNCR peptide is aligned with SEQ ID NO: 1 at all cysteine residues, and wherein the CaNCR peptide variant has at least 50% sequence identity to SEQ ID NO: 1, 8, 15, or 22 are provided.

Compositions comprising the aforementioned CaNCR peptide variants and an agriculturally, pharmaceutically, or veterinarily acceptable carrier, diluent, or excipient are provided.

Plant part comprising at least a partial coating and/or infiltrate comprising the aforementioned compositions or a processed plant product obtained therefrom comprising the compositions are provided.

Methods for preventing or reducing crop damage by a plant pathogenic microbe comprising the step of contacting a plant, a plant seed, or other part of said plant with an effective amount of the aforementioned compositions are provided.

Medical devices comprising the device and the aforementioned compositions, wherein the device comprises at least one surface that is topically coated and/or impregnated with the composition are also provided.

Methods for treating, preventing, or inhibiting a microbial or yeast infection in a subject in need thereof comprising administering to said subject an effective amount of the aforementioned compositions are also provided.

Polynucleotides comprising a DNA or RNA molecule encoding any of the aforementioned CaNCR peptide variants are provided.

Cells, plants, and processed plant products comprising the aforementioned polynucleotides are provided.

Use of any of the aforementioned polynucleotides or edited genomes, transformed or edited host cells, transgenic or genetically edited plants, transgenic or genetically edited plant parts, processed plant products, peptides, transgenic or genetically edited seed, medical devices, or compositions to inhibit growth of a susceptible microbial species is also provided. In certain embodiments of any of the aforementioned uses, the susceptible microbial species is a Fusarium sp., Alternaria sp., Verticillium sp., Phytophthora sp., Colletotrichum sp., Botrytis cinerea, Cercospora sp., Phakopsora sp. Rhizoctonia sp., Sclerotinia sp., Pythium sp., or Puccinia sp. or is a human and animal microbial pathogen that is an Aspergillus sp., Fusarium sp., Candida sp., Histoplasma sp., Paracoccidioides sp., Sporothrix sp., Blastomyces sp., Coccidioides sp., Geomyces sp., Trichophyton sp., or Malassezia sp. Use of any of any of the aforementioned compositions in a method of treating, preventing, or inhibiting microbial or yeast infection in a subject in need thereof are provided. Use of any of the aforementioned first antimicrobial peptide in the manufacture of a medicament or composition for inhibiting microbial infection in a subject in need thereof are also provided.

### BRIEF DESCRIPTION OF THE FIGURES

FIG. 1 shows an alignment of the CaNCR7 wild type peptide (SEQ ID NO: 8), the CaNCR14 wild type peptide (SEQ ID NO: 15), and the CaNCR15 wild type peptide (SEQ ID NO: 22) with the full length CaNCR13 wild-type reference peptide of SEQ ID NO: 1 where the CaNCR7, CaNCR14, and CaNCR15 peptides are aligned with SEQ ID NO: 1 at all cysteine residues.

FIG. 2 shows an alignment of the CaNCR13_V1, _V2, _V3, _V4 (SEQ ID NO: 2, 3, 4, 5, respectively), the CaNCR7_V1, _V2, _V3, _V4 (SEQ ID NO: 9, 10, 11, and 12, respectively), the CaNCR14_V1, _V2, _V3, _V4 (SEQ ID NO: 16, 17, 18, 19, respectively), and the CaNCR15_V1,_V2,_V3,_V4 (SEQ ID NO: 23, 24, 25, 26, respectively) CaNCR peptide variants with the full length CaNCR13 wild-type reference peptide of SEQ ID NO: 1, where the CaNCR peptide variants are aligned with SEQ ID NO: 1 at all cysteine residues. Also shown at the bottom of the alignment is the variant core consensus sequence of SEQ ID NO: 30.

FIG. 3 shows an alignment of the CaNCR13_V1, _V2,_V3, _V4, _V5, _V6, _V7, _V8, V9, _V10, _V11,_V12,_V13, _V14, _V15, and _V16 peptide variants (SEQ ID NO: 2, 3, 4, 5, 34, 35, 36, 37, 46, 47, 48, 49, 50, 51, 52, and 53, respectively) where the CaNCR peptide variants are aligned with CaNCR13 (SEQ ID NO: 1) at all cysteine residues. Also shown at the bottom of the alignment is the variant core consensus sequences of SEQ ID NO: 29 and 30.

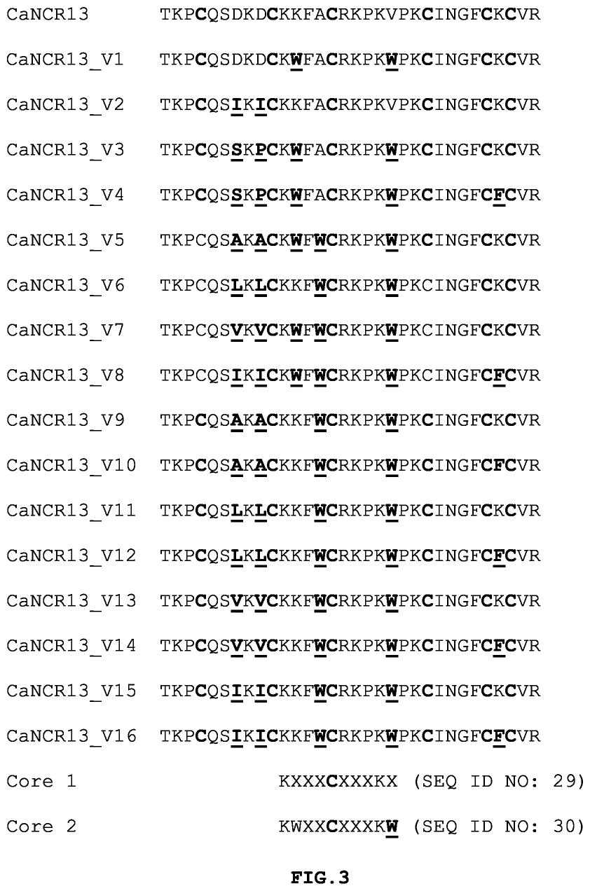

### DETAILED DESCRIPTION

### Definitions

Amino acid residues in polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

Where a term is provided in the singular, other embodiments described by the plural of that term are also provided.

As used herein, a polynucleotide is said to be “endogenous” to a given cell when it is found in a naturally occurring form and genomic location in the cell.

The phrases “antimicrobial peptide” as used herein refer to peptides which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat).

As used herein, the terms “acidic” or “anionic” are used interchangeably to refer to amino acids such as aspartic acid and glutamic acid.

As used herein, the phrase “CaNCR peptide” refers to CaNCR13 peptide of SEQ ID NO: 1, a CaNCR7 peptide of SEQ ID NO: 8, a CaNCR14 peptide of SEQ ID NO: 15, or a CaNCR15 peptide of SEQ ID NO: 22.

As used herein, the terms “basic” and “cationic” are used interchangeably to refer to amino acids such as arginine, histidine, and lysine.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “combating microbial damage”, “combating or controlling microbial damage” or “controlling microbial damage” as used herein refer to reduction in damage to a crop plant or crop plant product due to infection by a microbial pathogen. More generally, these phrases refer to reduction in the adverse effects caused by the presence of a pathogenic microbe in the crop plant. Adverse effects of microbial (e.g., fungal) growth are understood to include any type of plant tissue damage or necrosis, any type of plant yield reduction, any reduction in the value of the crop plant product, and/or production of undesirable microbial metabolites or microbial growth by-products including but not limited to mycotoxins.

The phrase “defensin peptide” is used herein to refer to a peptide comprising a conserved γ-core motif comprising a conserved GXCX3-9C sequence, where X is any amino acid residue. Defensin peptides include proteins that are antimicrobial, that can bind phospholipids, that can permeabilize plasma membranes, that can bind sphingolipids, or that exhibit any combination of those properties. A defensin peptide can be naturally occurring or non-naturally occurring (e.g., synthetic and/or chimeric).

As used herein, the terms “edit,” “editing”, and “edited” and the like refer to processes or products where insertions, deletions, and/or nucleotide substitutions are introduced into a genome. Such processes include methods of inducing homology directed repair and/or non-homologous end joining of one or more sites in the genome.

The term “endoproteinase” is used herein to refer to a peptidase capable of cleaving a peptide bond between two internal amino acid residues in a peptide sequence. Endoproteinases can also be referred to as “endoproteases” or “endopeptidases.” The proteolytic activity of an endoproteinase, endoprotease, or endopeptidase is thus different that the proteolytic activity of an “exopeptidase”, which cleaves peptide bonds of terminal amino acid residues in a peptide.

The phrases “genetically edited plant” or “edited plant” are used herein to refer to a plant comprising one or more nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′UTR, and 3′UTR), translation, or a combination thereof as well as polynucleotides encoding CaNCR peptide variants, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding CaNCR peptide variants, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding CaNCR peptide variants, spacer peptides, or localization can be heterologous to another polynucleotide when the polynucleotides are not operably linked to one another in a naturally occurring cell. Heterologous peptides include peptides that are not found in a cell or organism as the cell or organism occurs in nature. As such, heterologous peptides include peptides that are localized in a subcellular location, extracellular location, or expressed in a tissue that is distinct from the subcellular location, extracellular location, or tissue where the peptide or protein is found in a cell or organism as it occurs in nature. Heterologous polynucleotides include polynucleotides that are not found in a cell or organism as the cell or organism occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and in certain embodiments can perform the same biological function in either the same or a different organism.

The phrases “inhibiting growth of a plant pathogenic microbe,” “inhibit microbial growth” and the like as used herein refers to methods that result in any measurable decrease in microbial growth, where microbial growth includes but is not limited to any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including but not limited to mycotoxins. As used herein, the phrase “inhibition of microbial growth” and the like, unless otherwise specified, can include inhibition in a plant, human or animal.

As used herein, the phrase “junction sequence”, when used in the context of a CaNCR peptide variant multimer, refers to an amino acid sequence of about six residues where at least three (3) residues are contributed by a spacer peptide and at least three (3) residues are contributed by a CaNCR peptide variant. In certain embodiments, 3 amino acids at the N-terminus of the junction sequence are contributed by the final 3 C-terminal residues of the CaNCR peptide variant sequence and 3 amino acids at the C-terminus of the junction sequence are contributed by the first 3 N-terminal residues of the spacer peptide sequence. In certain embodiments, 3 amino acids at the N-terminus of the junction sequence are contributed by the final 3 C-terminal residues of the spacer peptide sequence and 3 amino acids at the C-terminus of the junction sequence are contributed by the first 3 N-terminal residues of the CaNCR peptide variant sequence.

As used herein, the phrase “linker peptide” refers to any peptide that joins a CaNCR peptide variant to another peptide, including a CaNCR peptide variant or defensin peptide, in a protein. In certain embodiments, a linker peptide can be susceptible to cleavage by an endoproteinase. In certain alternative embodiments, a linker peptide can be a spacer peptide that is resistant to endoproteinase cleavage. One embodiment where a linker peptide can be (e.g., function as) a spacer peptide is when the linker peptide that joins a CaNCR peptide variant to another peptide is localized in an extracellular or sub-cellular location that is deficient in endogenous endoproteinases that can cleave that linker peptide. One embodiment where a linker peptide can be (e.g., function as) a spacer peptide is when the linker peptide is joined to one or more heterologous CaNCR peptide variants that render the linker peptide resistant to endoproteinase cleavage. Another embodiment where a linker peptide can be (e.g., function as) a spacer peptide is when the linker peptide is joined to CaNCR peptide variant(s) via a heterologous junction sequence or sequences that render the linker peptide resistant to endoproteinase cleavage. A linker peptide can be naturally occurring or non-naturally occurring (e.g., synthetic).

As used herein, the phrase “linker peptide that is susceptible to cleavage by a endoproteinase” when used in the context of a linker peptide sequence that joins two CaNCR peptide variants in a single encoded protein, refers to a linker peptide sequence that permits less than 50% of CaNCR peptide variant containing protein in a transgenic or genetically edited organism or cell, an extracellular space of the organism or cell, a sub-cellular location of the organism or cell, or any combination thereof to accumulate as a protein comprising the linker peptide and both peptides that are covalently linked thereto. The phrase “linker peptide that is susceptible to cleavage by a plant endoproteinase” when used in the context of a linker peptide sequence that joins a CaNCR peptide variant to another peptide in a single encoded protein, refers to a linker peptide sequence that permits less than 50% of CaNCR peptide variant-containing protein in a transgenic or genetically edited plant or cell, an extracellular space of the plant or cell, a sub-cellular location of the plant or cell, or any combination thereof to accumulate as a protein comprising the linker peptide and both peptides that are covalently linked thereto.

In certain embodiments, the endoproteinase is an endogenous plant, yeast, or mammalian endoproteinase.

As used herein, the terms “microbe”, “microbes” and “microbial” are used to refer to bacteria, fungi (including yeast), and oomycetes.

As used herein, the term “CaNCR peptide variant” refers to any peptide with antimicrobial activity comprising one or more non-conservative amino acid substitutions in a CaNCR peptide. A CaNCR peptide variant can, in addition to such non-conservative substitutions, further comprise a peptide having conservative amino acid substitutions, deletions of one to five amino acids from the N-terminus, and internal deletions of one or more amino acid residues in a CaNCR peptide variant peptide provided herein.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5′untranslated sequence associated with the promoter and the coding sequence is linked such that the resulting translation product is in frame with the translational open reading frame that encodes the protein. Nucleic acid sequences that can be operably linked include sequences that provide gene expression functions (e.g., gene expression elements such as promoters, 5′ untranslated regions, introns, protein coding regions, 3′untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, linker, or other peptide, “operably linked” means that the peptide is connected to the polyprotein sequence(s) of interest such that it provides a function. Functions of a localization peptide include localization of a protein or peptide of interest (e.g., a CaNCR peptide variant or CaNCR peptide variant multimer) to an extracellular space or subcellular compartment. Functions of a spacer peptide include linkage of two peptides of interest (e.g., two CaNCR peptide variants) such that the peptides will be expressed as a single protein (e.g., a CaNCR peptide variant multimer).

The phrases “percent identity” or “sequence identity” as used herein refer to the number of elements (i.e., amino acids or nucleotides) in a sequence that are identical within a defined length of two DNA, RNA or protein segments in an alignment resulting in the maximal number of identical elements, and is calculated by dividing the number of identical elements by the total number of elements in the defined length of the aligned segments and multiplying by 100.

As used herein, the phrase “resistant to cleavage by an endoproteinase” when used in the context of a spacer peptide sequence that joins at least one CaNCR peptide variant and another peptide (including an identical or distinct CaNCR peptide variant) in a single encoded CaNCR peptide variant multimer polypeptide, refers to a spacer peptide sequence that permits more than 50%, 60%, 70%, 80%, 90%, or 95% of the polypeptide in a transgenic or genetically edited organism, cell, extracellular space of the organism or cell, sub-cellular location of the organism or cell, or any combination thereof to accumulate as a CaNCR peptide variant multimer that comprises the spacer peptide, the CaNCR peptide variant and other peptide that is covalently linked thereto. The phrase “resistant to cleavage by a plant endoproteinase”, when used in the context of a spacer peptide sequence that joins at least one CaNCR peptide variant to another peptide (including a CaNCR peptide variant) in a single encoded protein, refers to a spacer peptide sequence that permits more than 50%, 60%, 70%, 80%, 90%, or 95% of the CaNCR peptide variant multimer in a transgenic or genetically edited plant or plant cell, an extracellular space of the plant or cell, a sub-cellular location of the plant or cell, or any combination thereof to accumulate as a CaNCR peptide variant multimer that comprises the spacer peptide and the CaNCR peptide variant and other peptide (including a CaNCR peptide variant) that are covalently linked thereto.

As used herein, the phrase “spacer peptide” refers to any peptide that joins CaNCR peptide variant to another peptide in a protein that is resistant to cleavage by an endoproteinase. In certain embodiments, the endoproteinase is an endogenous plant, yeast, or mammalian endoproteinase. A spacer peptide can be naturally occurring or non-naturally occurring (e.g., synthetic).

The terms “susceptible microbe (or microbes),” “susceptible microbial infection” and the like refer to microbes that infect plants, or human or animal patients or subjects, or microbial infections thereof, that are subjection to inhibition of microbial growth by the CaNCR peptide variants or CaNCR peptide variant multimers disclosed herein.

The phrase “transgenic” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### FURTHER DESCRIPTION

Substituted variants of antimicrobial nodule specific cysteine rich peptides referred to as CaNCR peptide variants are provided herein. In certain embodiments, the CaNCR peptide variants are linked by a spacer peptide that is resistant to plant or other endoproteinase cleavage to form a CaNCR peptide variant multimer comprising two CaNCR peptide variants. The antimicrobial peptides can be applied directly to a plant, applied to a plant in the form of microorganisms that produce the CaNCR peptide variant or CaNCR peptide variant multimer, or the plants can be genetically edited to produce the CaNCR peptide variant or CaNCR peptide variant multimer. The present disclosure also relates to recombinant or edited polynucleotides, microorganisms and plants transformed with the recombinant nucleic acids, plants comprising genetically edited nuclear or plastid genomes encoding the CaNCR peptide variants or CaNCR peptide variant multimers and compositions comprising the CaNCR peptide variants or CaNCR peptide variant multimers useful in controlling pathogenic microbes including, but not limited to, plant, animal, and human pathogenic microbes.

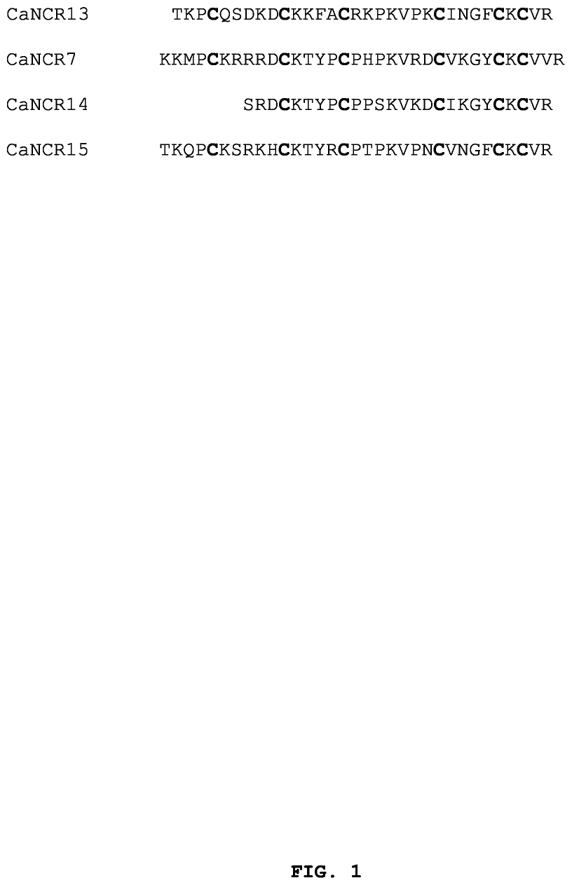

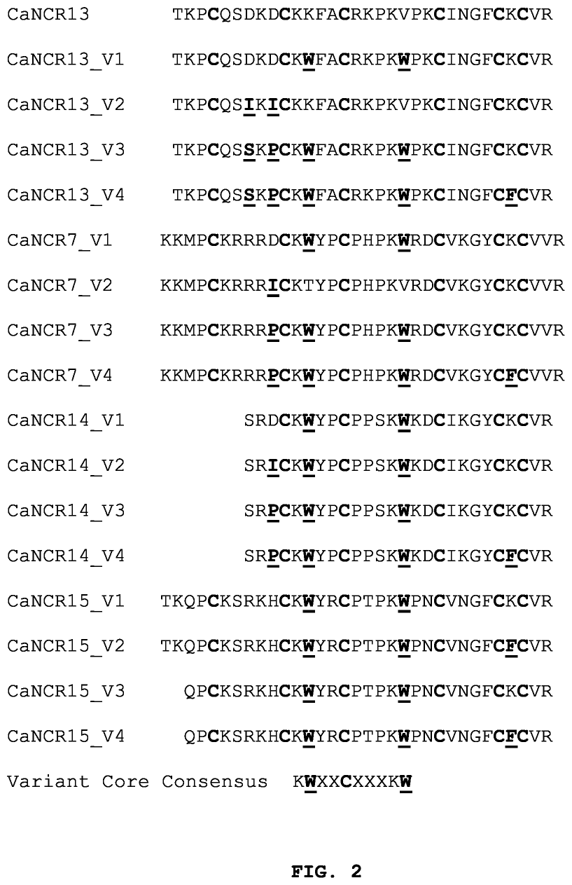

CaNCR peptide variants provided can comprise one or more non-conservative amino residue substitutions. In certain embodiments, CaNCR peptide variants comprise amino acid substitutions in a CaNCR peptide (e.g., SEQ ID NO: 1, 8, 15, 22, or a conservatively substituted variant thereof) corresponding to a D7I substitution, a D7S substitution, a D7A substitution, D7V substitution, D7L substitution, a D9A substitution, D9V substitution, a D9L substitution, a D9I substitution, a D9P substitution, a K12W substitution, a K29F substitution, or any combination of said substitutions in a full length reference peptide of SEQ ID NO: 1 when the CaNCR peptide is aligned with SEQ ID NO: 1 at all cysteine residues. Alignments of a CaNCR peptide to a CaNCR reference peptide are depicted in FIG. 1. Alignments of substituted CaNCR peptides comprising CaNCR peptide variants to a CaNCR reference peptide of SEQ ID NO: 1 are depicted in FIG. 2 and FIG. 6. Conservatively substituted variants of SEQ ID NO: 1, 8, and 22 which can be further or alternatively substituted at positions corresponding to a D7I substitution, a D7S substitution, a D7A substitution, a D9I substitution, a D9P substitution, a D9A substitution, D7V substitution, D7L substitution, a D9A substitution, D9V substitution, a D9L substitution, a K12W substitution, a K29F substitution, or any combination of said substitutions in a full length reference peptide of SEQ ID NO: 1 include conservatively substituted variants of SEQ ID NO: 1, 8, and 22 set forth in Table 1A, 1B, and 1C and FIG. 6. In certain embodiments, the CaNCR peptide variants can include conservative amino acid substitution corresponding to an A14W and/or a V20W substitution in a CaNCR reference peptide of SEQ ID NO: 1. In certain embodiments, CaNCR peptide variants comprising one or more of the aforementioned substitutions in a CaNCR peptide of SEQ ID NO: 1, 8, 15, 22 or a conservatively substituted variant thereof will comprise or conserve amino acid motifs which include SEQ ID NO: 6, 7, 13, 14, 20, 21, 27, or 28. In certain embodiments, CaNCR peptide variants comprising certain aforementioned substitutions in a CaNCR peptide will comprise or conserve amino acid motifs which include: (i) SEQ ID NO: 6 or 7 (e.g., in CaNCR peptide variants comprising aforementioned non-conservative substitutions in SEQ ID NO: 1 or in conservatively substituted variants thereof); (ii) SEQ ID NO: 13 or 14 (e.g., in CaNCR peptide variants comprising aforementioned non-conservative substitutions in SEQ ID NO: 8 or in conservatively substituted variants thereof); (iii) SEQ ID NO: 20 or 21 (e.g., in CaNCR peptide variants comprising aforementioned non-conservative substitutions in SEQ ID NO: 15 or in conservatively substituted variants thereof); and (iv) SEQ ID NO: 27 or 28 (e.g., in CaNCR peptide variants comprising aforementioned non-conservative substitutions in SEQ ID NO: 22 or in conservatively substituted variants thereof). In certain embodiments, any of the aforementioned CaNCR peptide variants can comprise a polypeptide sequence comprising one or more of the non-conservative amino acid substitutions, wherein the polypeptide has at least 50%, 55%, 60%, 70%, 80%, 84%, 87%, 90%, 93%, or 96% sequence identity across the entire length of SEQ ID NO: 1, 8, 15, or 22, optionally wherein the polypeptide can optionally further comprise any of the aforementioned conservative amino acid residue substitutions. In certain embodiments, any of the aforementioned CaNCR peptide variants can comprise a polypeptide having at least 50%, 55%, 60%, 70%, 80%, 84%, 87%, 90%, 93%, 96%, or 100% sequence identity to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53. In certain embodiments any of the aforementioned CaNCR peptide variants can comprise a polypeptide sequence comprising one or more of the non-conservative amino acid substitutions and at least 50%, 55%, 60%, 70%, 80%, 84%, 87%, 90%, 93%, 96%, 97%, 98%, or 99% sequence identity to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53.

CaNCR peptide variants provided and used in various embodiments disclosed herein can comprise one or more of the following structural features.

In certain embodiments, a structural feature in certain CaNCR peptide variants is the presence of a core consensus sequence KX2X3X4CX6X7X8KX10 (SEQ ID NO: 29), where X2 is K, T, or W, wherein X3 is F or Y, wherein X4 is A, W, F, R, or P, wherein X6 is R or P, wherein X7 is K, H, T, or P, wherein X8 is P or S; and wherein X10 is V or W or a variant thereof comprising an insertion, deletion, and/or substitution of one or more amino acid residues. In certain embodiments, in the core consensus sequence (SEQ ID NO: 29) is located in a CaNCR peptide variant at positions corresponding to K11 to V20 in a full-length reference peptide of SEQ ID NO: 1 when the CaNCR peptide variant is aligned with SEQ ID NO: 1 at all cysteine residues. Such an alignment of certain CaNCR peptide variants with a SEQ ID NO: 1 reference peptide is depicted in FIG. 2.

In certain embodiments, a structural feature in certain CaNCR peptide variants is the presence of a CaNCR peptide variant core consensus sequence KWX3X4CX6X7X8KW (SEQ ID NO: 30), where X3 is F or Y, where X4 is A, W, F, R, or P, where X6 is R or P, where X7 is K, H, T, or P, and where X8 is P or S, or a variant thereof comprising an insertion, deletion, and/or substitution of one or more amino acid residues other than the two W residues. In certain embodiments, in the CaNCR peptide variant core consensus sequence (SEQ ID NO: 30) is located in a CaNCR peptide variant at positions corresponding to K11 to V20 in a full-length reference peptide of SEQ ID NO: 1 when the CaNCR peptide variant is aligned with SEQ ID NO: 1 at all cysteine residues. Such an alignment of certain CaNCR peptide variants and the CaNCR peptide variant core consensus sequence with a SEQ ID NO: 1 reference peptide is depicted in FIG. 2.

In certain embodiments, a structural feature of the CaNCR peptide variants is a net positive charge at neutral pH. In certain embodiments, the CaNCR peptide variants will have a net positive charge at neutral pH of at least +5, +6, +7, +8, +9, or +10. In certain embodiments, the CaNCR peptide variants will have a net positive charge at neutral pH of at least +4, +5, +6, +7, +8, +9, or +10 to +12, +13, +14, or +15. In certain embodiments, such net positive charges in CaNCR peptide variants can be achieved by methods that include: (i) maintaining cationic (basic) amino acid residues found in CaNCR peptide variants including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 or substituting such residues with another cationic amino acid residue (e.g., with a cationic amino acid substitution corresponding to a cationic amino substitution in a SEQ ID NO: 1, 8, or 22 reference peptide as shown in Tables 1A, 1B and 1C, respectively); (ii) substituting anionic or polar amino acid residues found in CaNCR peptide variants including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 with a basic amino acid residue; or a combination of (i) and (ii). In certain embodiments, such net positive charges in CaNCR peptide variants can be achieved by preferentially selecting or substituting a cationic amino acid residue at variable positions in the CaNCR peptide variant that correspond to a variable position of SEQ ID NO: 29 or 30. For example, a cationic amino acid can be preferentially selected or substituted for any of the variable amino acids at positions X₂, X₄, or X₇ of SEQ ID NO: 29 or 30.

In certain embodiments, a structural feature of the CaNCR peptide variants is a significant percentage of hydrophobic amino acid residues. In certain embodiments, the CaNCR peptide variants will comprise at least about 25%, 26%, 28% 30%, 32%, 34%, 36%, 37%, or 38% hydrophobic amino acid residues. In certain embodiments, the CaNCR peptide variants will comprise at least about 25%, 26%, 28% 30%, 32%, 34%, or 36% to 37%, 38%, 40%, 42%, or 45% hydrophobic amino acid residues. In certain embodiments, such percentages of hydrophobic amino acids in CaNCR peptide variants can be achieved by methods that include: (i) maintaining hydrophobic amino acid residues found in CaNCR peptide variants including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 or substituting such residues with another hydrophobic amino acid residue or neutral polar amino acid residue (e.g., with a hydrophobic amino acid substitution corresponding to a hydrophobic amino substitution in a SEQ ID NO: 1, 8, or 22 reference peptide as shown in Tables 1A, 1B and 1C, respectively); (ii) substituting polar amino acid residues found in CaNCR peptide variants including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 with a hydrophobic amino acid residue; (iii) substituting neutral polar amino acids for hydrophobic amino acids; or a combination of (i), (ii), and (iii). Examples of such substitutions of hydrophobic amino acid residues in certain CaNCR peptide variants include those corresponding to substitutions of certain hydrophobic residues set forth in the CaNCR peptides of SEQ ID NO: 1, 8, and 22 shown in Tables 1A, 1B and 1C, respectively. In certain embodiments, such percentages of hydrophobic amino acids in CaNCR peptide variants can be achieved by preferentially selecting or substituting a hydrophobic amino acid residue at variable positions in the CaNCR peptide variant that correspond to a variable position of SEQ ID NO: 29 or 30. For example, a hydrophobic amino acid can be preferentially selected or substituted for variable amino acids X₂, X₄, X₆, X₇, X₈, or X₁₀ of SEQ ID NO: 29 or 30.

TABLE 1A
NCR3 (SEQ ID NO: 1)
Cationic Amino Acids

| | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| T | K | P | C | Q | S | D | K | D | C | K | K | F | A | C | R | K | P | K | V | P | K | C | I | N | G | F | C | K | C | V | R |
| | R | | | | | | R | | | R | R | | | | K | R | | R | | | R | | | | | | | R | | | K |
| | H | | | | | | H | | | H | H | | | | H | H | | H | | | H | | | | | | | H | | | H |

Anionic Amino Acids

| | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| T | K | P | C | Q | S | D | K | D | C | K | K | F | A | C | R | K | P | K | V | P | K | C | I | N | G | F | C | K | C | V | R |
| | | | | | | E | | E | | | | | | | | | | | | | | | | | | | | | | | |

Hydrophobic Amino Acids

| | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| T | K | P | C | Q | S | D | K | D | C | K | K | F | A | C | R | K | P | K | V | P | K | C | I | N | G | F | C | K | C | V | R |
| | | | | | | | | | | | | W | W | | | | | | W | | | | W | | | W | | | | W | |
| | | | | | | | | | | | | I | I | | | | | | I | | | | V | | | I | | | | I | |
| | | | | | | | | | | | | M | M | | | | | | M | | | | M | | | M | | | | M | |
| | | | | | | | | | | | | V | V | | | | | | F | | | | F | | | V | | | | F | |
| | | | | | | | | | | | | L | L | | | | | | L | | | | L | | | L | | | | L | |
| | | | | | | | | | | | | A | F | | | | | | A | | | | A | | | A | | | | A | |

TABLE 1B
NCR7 (SEQ ID NO: 8)
Cationic Amino Acids

| | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|
| K | K | M | P | C | K | R | R | R | D | C | K | T | Y | P | C | P | H | P | K | V | R | D | C | V | K | G | Y | C | K | C | V | V | R |
| R | | | | | R | K | K | K | | | R | | | | | | | | R | | K | | | | R | | | | R | | | | R |
| H | | | | | H | H | H | H | | | H | | | | | | | | H | | H | | | | H | | | | H | | | | H |

Anionic Amino Acids

| | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|
| K | K | M | P | C | K | R | R | R | D | C | K | T | Y | P | C | P | H | P | K | V | R | D | C | V | K | G | Y | C | K | C | V | V | R |
| | | | | | | | | | E | | | | | | | | | | | | | E | | | | | | | | | | | |

Hydrophobic Amino Acids

| | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|
| K | K | M | P | C | K | R | R | R | D | C | K | T | Y | P | C | P | H | P | K | V | R | D | C | V | K | G | Y | C | K | C | V | V | R |
| | | W | | | | | | | | | | | | | | | | | | W | | | | W | | | | | | | W | W | |
| | | F | | | | | | | | | | | | | | | | | | F | | | | F | | | | | | | F | F | |
| | | I | | | | | | | | | | | | | | | | | | I | | | | I | | | | | | | I | I | |
| | | V | | | | | | | | | | | | | | | | | | M | | | | M | | | | | | | M | M | |
| | | L | | | | | | | | | | | | | | | | | | L | | | | L | | | | | | | L | L | |
| | | A | | | | | | | | | | | | | | | | | | A | | | | A | | | | | | | A | A | |

TABLE 1C
NCR15 (SEQ ID NO: 22)
Cationic Amino Acids

| | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|
| T | K | Q | P | C | K | S | R | K | H | C | K | T | Y | R | C | P | T | P | K | V | P | N | C | V | N | G | F | C | K | C | V | R |
| | R | | | | R | | K | R | R | | R | | | K | | | | | R | | | | | | | | | | R | | | K |
| | H | | | | H | | H | H | K | | H | | | H | | | | | H | | | | | | | | | | H | | | H |

Hydrophobic Amino Acids

| | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|
| T | K | Q | P | C | K | S | R | K | H | C | K | T | Y | R | C | P | T | P | K | V | P | N | C | V | N | G | F | C | K | C | V | R |
| | | | | | | | | | | | | | | | | | | | | W | | | | W | | | W | | | | W | |
| | | | | | | | | | | | | | | | | | | | | F | | | | F | | | V | | | | F | |
| | | | | | | | | | | | | | | | | | | | | I | | | | I | | | I | | | | I | |
| | | | | | | | | | | | | | | | | | | | | M | | | | M | | | M | | | | M | |
| | | | | | | | | | | | | | | | | | | | | L | | | | L | | | L | | | | L | |
| | | | | | | | | | | | | | | | | | | | | A | | | | A | | | A | | | | A | |

In certain embodiments, a structural feature of CaNCR peptide variants is presence of one or more of the 6 conserved cysteine residues set forth in SEQ ID NO: 1, 8, 15, or 22. Such conserved cysteine residues are depicted in FIG. 2. In certain embodiments, at least two, at least four, or at least six of the conserved cysteine residues in CaNCR peptide variants provided herein can also comprise substitutions of one or more of the conserved cysteine residues (e.g., C1, C2, C3, C4, C5, and C6 in SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 where the cysteine residue closest to the amino terminus is C1 and the cysteine residue closest to the carboxy terminus is C6). In certain embodiments, one or more of the conserved cysteine residues can be substituted with another amino acid residue including a glycine, serine, threonine, cysteine, cystine, tyrosine, asparagine, or glutamine residue. In certain embodiments, one or more of the conserved cysteine residues can be substituted with a serine residue. While not being limited by theory, it is believed that CaNCR peptide variants with substitutions of cysteine residues and that lack one or more disulfide linkages may be desirable for use in transgenic or gene edited plants that are ultimately used as animal feed or as food for human consumption as such variants are predicted to be more readily digested by animals or humans that consume the plant products. Such CaNCR peptide variants that have shorter half-lives in the digestive tracts of animals or humans are in theory anticipated to have less potential to become food allergens.

In any of the aforementioned embodiments, the CaNCR peptide variant(s) can comprise an amino acid sequence that is at least 60%, 70%, 75%, 80%, 85%, 90%, 95%, 98%, 99%, or 100% identical to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 or a variant thereof wherein one or more of the hydrophobic, basic, and/or acidic amino acid residues of SEQ ID NO: 2, 3, 4, 5, 9, 10, 11, 12, 16, 17, 18, 19, 23, 24, 25, 26, 34, 35, 36, or 37 are conservatively substituted with other hydrophobic, basic, and/or acidic amino acid residues, respectively. In any of the aforementioned embodiments, the CaNCR peptide variant(s) can also comprise an amino acid sequence that is at least 70%, 75%, 80%, 85%, 90%, 95%, 98%, or 99%, identical to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 wherein one or more of the hydrophobic, basic, and/or acidic amino acid residues of SEQ ID NO: 1, 2, 3, 4, 5, 9, 10, 11, 12, 16, 17, 18, 19, 23, 24, 25, 26, 34, 35, 36, 37, 46, 47, 48, 49, 50, 51, 52, or 53 are conservatively substituted with other hydrophobic, basic, and/or acidic amino acid residues, respectively.

In certain embodiments, spacer peptide domains that can be used to join CaNCR peptide variants to one another, to themselves, or to another antimicrobial peptide (e.g., a defensin peptide) obtain a CaNCR peptide variant multimer, where such CaNCR peptide variant multimers thus include CaNCR peptide variant dimers. Examples of spacer peptides that can be used as is or in a mutagenized form include the MtDef5 spacer peptide. Mutagenesis of any of the aforementioned spacer peptides can entail the insertion, deletion, or substitution of at least one, two, three, four, five, six, or seven amino acid residues in the linker peptide sequence that render the mutagenized linker peptide resistant to cleavage by a plant endoproteinase. Spacer peptides for use in the CaNCR peptide variant multimers can also be obtained from multimeric- or multi-domain proteins that do not contain defensin or other antimicrobial peptides. Such peptide linker sequences that join peptides in multimeric or multi-domain proteins have been disclosed (Argos, 1990; George R A, Heringa (2002). Examples of suitable peptide sequences from multimeric or multi-domain proteins that can be used as spacer domains include, but are not limited, immunoglobulin hinge regions from immunoglobulins, a linker between the lipoyl and E3 binding domain in pyruvate dehydrogenase (Turner et al., 1993), a linker between the central and C-terminal domains in cysteine proteinase (P9; Mottram et al., 1989), and functional variants thereof. Spacer peptides for use in the CaNCR peptide variant multimers can also be wholly or partially synthetic peptide sequences. Such synthetic spacer peptides are designed to provide for a flexible linkage between a CaNCR peptide variant and other peptides (e.g., the same or different CaNCR peptide variant peptides or defensins) and to be resistant to cleavage by endogenous plant endoproteinases. In certain embodiments, the length of the synthetic spacer peptide can be between about 3, 4, 8, 10, 12, or 16 and about 20, 24, 28, 30, 40, or 50 amino acid residues in length. In certain embodiments, the synthetic spacer peptide can comprise a glycine-rich or glycine/serine containing peptide sequence. Such sequences can include a (Gly4)n sequence (SEQ ID NO: 31), a (Gly4Ser)n sequence (SEQ ID NO: 32), a Ser (Gly4Ser)n sequence (SEQ ID NO: 33), combinations thereof, and variants thereof, wherein n is a positive integer equal to 1, 2, 3, 4, 5, 6, 7, 8, 9 or 10. In certain embodiments, such glycine-rich or glycine/serine containing synthetic peptide sequences can also contain threonine and/or alanine residues for flexibility as well as polar lysine and/or glutamine residues. Additional synthetic linker sequences that can be used as spacer peptides include combinations thereof, and variants thereof. Such variants of synthetic linker sequences include insertions, deletions, and substitutions of amino acid residues. Variants of any of the aforementioned synthetic peptide spacers also include, but are not limited, to insertions and/or substitutions of one or more residues that frequently occur in peptides that join domains in proteins such as proline, arginine, phenylalanine, threonine, glutamine, and combinations thereof. In certain embodiments, such glycine-rich, glycine/serine containing peptide sequence, or other synthetic peptide spacer sequence can be used to mutagenize a linker peptide sequence. In certain embodiments, mutagenesis of a linker peptide sequence by insertion and/or substitution of a glycine-rich or glycine/serine containing peptide sequence can be used to disrupt a peptide sequence recognized by a plant endoproteinase such as a set of diacidic and/or dibasic residues or a site that is cleaved by a cysteine, serine, threonine, metallo-, or aspartic plant endoproteinase. The composition and design of peptides suitable for flexible linkage of protein domains described in the literature (Chen et al., 2013) can be adapted for use as spacer peptides in the CaNCR peptide variant multimers provided herein. Spacer peptides useful for joining defensin monomers described in US Patent Appl. Pub. 20190194268 and US Patent Appl. Pub. 20190185877, which are each incorporated herein by reference in their entireties, can also be used to join CaNCR peptide variants disclosed herein to other CaNCR peptide variants, defensins, antimicrobial peptides, or other peptides to form CaNCR peptide variant multimers.

Since the CaNCR peptide variants can be joined to one another or other peptides in CaNCR peptide variant multimers, the spacer peptide sequences, and the junction sequences formed by joining either the amino- or carboxy-terminus of a CaNCR peptide variant to a spacer peptide are in certain embodiments also designed or engineered to be free of amino acid sequences that are susceptible to cleavage by plant or other endoproteinases. In designing CaNCR peptide variant multimers for expression in plant or other hosts including bacteria, yeast, mammalian cells, and the like, the spacer peptide and junction sequences will typically lack diacidic (aspartyl residues, glutamyl residues, and any combination thereof), dibasic (arginine residues, lysine residues, and any combination thereof), or combinations of diacidic and dibasic residues in certain embodiments provided herein. Spacer peptide and junction sequences will typically be resistant to cleavage by at least one of a cysteine, serine, threonine, metallo-, or aspartic plant endoproteinase in certain embodiments provided herein. Amino acid sequences identified as plant endoproteinase substrates (Tsiatsiani et al., 2012) will also typically be absent from spacer peptide and junction sequences in certain embodiments provided herein.

In certain embodiments, CaNCR peptide variant multimers provided herein can comprise a spacer peptide or junction sequence that is susceptible to cleavage by a plant endoproteinase when the CaNCR peptide variant multimer is expressed in a plant, plant cell, yeast cell, or mammalian cell in a manner that that will prevent such cleavage. In one such embodiment, the CaNCR peptide variant multimer that comprises a spacer peptide or junction sequence that is susceptible to cleavage by a plant endoproteinase is targeted to an extracellular or sub-cellular compartment where activity of that plant endoproteinase reduced or absent. In certain embodiments where the spacer peptide is resistant to cleavage by endoproteinases in the plant cell cytoplasm, the CaNCR peptide variant dimers can be expressed in the cytoplasm by expressing a CaNCR peptide variant dimer that lacks any targeting signals. In certain embodiments, a CaNCR peptide variant multimer that comprises a spacer peptide or junction sequence that is susceptible to cleavage by a vacuolar plant endoproteinase is targeted to either the apoplast, plastids, mitochondria, or endoplasmic reticulum by operable linkage of suitable localization peptides to that CaNCR peptide variant multimer and/or by removal of any vacuolar localization signal that could have been associated with a given CaNCR peptide variant or protein. In certain embodiments, a CaNCR peptide variant multimer that comprises a spacer peptide or junction sequence that is susceptible to cleavage by a plastidic plant endoproteinase is targeted to either the apoplast, mitochondria, endoplasmic reticulum, or vacuole by operable linkage of suitable localization peptides to that CaNCR peptide variant multimer. In certain embodiments, a CaNCR peptide variant multimer that comprises a spacer peptide or junction sequence that is susceptible to cleavage by an apoplastic plant endoproteinase is targeted to either mitochondria, plastids, endoplasmic reticulum, or vacuole by operable linkage of suitable localization peptides to that CaNCR peptide variant. In certain embodiments, a CaNCR peptide variant multimer that comprises a spacer peptide or junction sequence that is susceptible to cleavage by a mitochondrial plant endoproteinase is targeted to an apoplastic space, plastids, endoplasmic reticulum, or vacuole by operable linkage of suitable localization peptides to that CaNCR peptide variant. Also provided herein are embodiments where a CaNCR peptide variant multimer that comprises one or more spacer peptides that are resistant to cleavage by a plant endoproteinase is targeted to the apoplast, plastids, mitochondria, vacuole, or endoplasmic reticulum.

A CaNCR peptide variant provided herein can be operably linked to another or the same CaNCR peptide variant, defensin, or antimicrobial peptide via a linker peptide sequence to obtain a CaNCR peptide variant multimer that is susceptible to cleavage by an endoproteinase, including a plant endoproteinase. In certain embodiments, the resultant CaNCR peptide variant multimer can be expressed in a cell such that the endoproteinase cleaves the CaNCR peptide variant multimer to provide the CaNCR peptide variant(s) and/or CaNCR peptide variant and defensin or another antimicrobial peptide. Such CaNCR peptide variant multimers can be provided in a cellular compartment (e.g., cytoplasm, mitochondria, plastid, vacuole, or endoplasmic reticulum) or extracellular space (i.e., to the apoplast) having an endoproteinase that cleaves the linker peptide. Cleavable linker peptides are disclosed in WO2014078900, Vasivarama and Kirti, 2013a, François et al., Vasivarama and Kirti, 2013b, and US Patent Appl. Pub. 20190194268 can be used in the CaNCR peptide variant multimers provided herein.

A variety of different CaNCR peptide variants can be used in the CaNCR peptide variant multimers comprising a CaNCR peptide variants and optionally another antimicrobial peptide. In certain embodiments, the CaNCR peptide variants in the CaNCR peptide variant multimer will be identical or related to one another such that the two peptides have at least 60%, 70%, 80%, 85%, 90%, 95%, 98%, 99%, or 100% sequence identity to one another. In certain embodiments, the CaNCR peptide variants will be distinct and have less than 50% identity to one another. In any of the aforementioned embodiments, the CaNCR peptide variant(s) can comprise an amino acid sequence that is at least 70%, 75%, 80%, 85%, 90%, 95%, 98%, 99%, or 100% identical to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53, a fragment thereof, or a chimera thereof. In any of the aforementioned embodiments, the CaNCR peptide variant(s) can comprise an amino acid sequence that is at least 70%, 75%, 80%, 85%, 90%, 95%, 98%, or 99% identical to SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53, wherein one or more of the hydrophobic, basic, and/or acidic amino acid residues of SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53, are substituted with other hydrophobic, basic, and/or acidic amino acid residues, respectively. In certain embodiments, a defensin peptide can be used in the CaNCR peptide variant multimer. In certain embodiments, the CaNCR peptide variant multimers can comprise a CaNCR peptide variant and an MtDef4, MtDef4 H33R, MsDef1, NaD1, TPP3, MtDef5, RsAFP2, DmAMP1, Psd1, HXL005, HXL008, HXL035, HXL036 defensin peptides and/or any defensin, spacer peptide, or linker peptide disclosed in US Patent Appl. Pub. 20190194268 and US Patent Appl. Pub. 20190185877, which are each incorporated herein by reference in their entireties.

In certain embodiments, one or more amino acids in any of the aforementioned or other variant CaNCR peptide variant sequences including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 are substituted with another amino acid(s), the charge and polarity of which is similar to that of the original amino acid, i.e., a conservative amino acid substitution. Substitutes for an amino acid within the CaNCR peptide variant sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (anionic; negatively charged) amino acids such as aspartic acid and glutamic acid; (2) basic (cationic; positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within CaNCR peptide variant sequences can be made by substituting one amino acid within one of these groups with another amino acid within the same group. Biologically functional equivalents of CaNCR peptide variants can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the CaNCR peptide variants. Certain semi-conservative substitutions in CaNCR peptide variants including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Semi-conservative substitutions of a hydrophobic amino acid residue with tyrosine residue are also provided. Biologically functional equivalents of CaNCR peptide variants can have 10 or fewer semi-conservative amino acid changes, seven or fewer semi-conservative amino acid changes, or five, four, three, two, or one semi-conservative amino acid changes.

Functional fragments of any of the aforementioned CaNCR peptide variants including SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 can comprise CaNCR peptide variants having amino terminal deletions, carboxy terminal deletions, internal deletions, or any combination thereof. In certain embodiments, the functional fragment can contain at least one, two, three, four, five, six, or seven or more amino acid residue deletions from the amino terminus, the carboxy terminus, an internal region, or any combination thereof. In certain embodiments, antimicrobial fragments of the CaNCR peptide variant can comprise at least about 22 to about 24, 25, 26, 27, or 28 amino acid residues of the C-terminus of the CaNCR peptide variant. In any of the aforementioned embodiments, the functional CaNCR peptide variant fragment can comprise a conserved core amino acid sequence motif set forth in SEQ ID NO: 6, 7, 13, 24, 20, 21, 27, 28, 29, or 30.

Chimeric CaNCR peptide variants comprising portions of any of the aforementioned or other CaNCR peptide variants can also be used either alone or in the CaNCR peptide variants multimers provided herein. Chimeric CaNCR peptide variants include peptides comprising an N-terminal fragment of a CaNCR peptide variant of SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 which is covalently linked to a C-terminal fragment of different CaNCR peptide variant of SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53.

In any of the aforementioned or other embodiments, the CaNCR peptide variant can also comprise one or more of the conserved cysteinyl residues set forth in SEQ ID NO: 1, 8, 15, or 22. Such conserved cysteine residues are illustrated herein in at least FIGS. 1 and 6. CaNCR peptide variant of SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 including any cysteinyl residues that are involved in formation of disulfide bridges. In certain embodiments, one or more cysteinyl residues in the CaNCR peptide variant can be substituted with a distinct amino acid residue. In certain embodiments, one or more cysteinyl residues in the CaNCR peptide variant can be substituted with a serine or threonine residue.

In certain embodiments, the permeability of a microbial plasma membrane treated with the CaNCR peptide variants comprising one or more non-conservative substitutions (e.g., a peptide comprising SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 or a functional fragment thereof) is increased in comparison to permeability of a microbial plasma membrane treated with a CaNCR peptide (e.g., a peptide comprising SEQ ID NO: 1, 8, 15, or 22). Membrane permeability can be measured by a variety of techniques that include dye uptake. Convenient dye uptake assays that can be used to assess changes in in membrane permeability include assays for uptake of Hoechst 33342 (H0342), rhodamine 123, SYTOX™ Green, and the like. These dyes enter into microbial cells only if their plasma membrane has been permeabilized by a defensin or another membrane-permeabilizing agent. Without seeking to be limited by theory, in certain embodiments it is believed that the CaNCR peptide variant can provide improved microbial inhibition by increasing the permeability of treated microbial membranes in comparison to microbial membranes treated with a CaNCR peptide.

In certain embodiments, the CaNCR peptide variant used alone or in a CaNCR peptide variant multimer are CaNCR peptide variants that exhibit binding to a phospholipid. In certain embodiments, CaNCR peptide variants provided herein can exhibit lower IC50 and/or MIC50 values against one or more microbial pathogens, improved binding to phospholipids, or any combination thereof in comparison to a reference peptide containing a CaNCR peptide. In certain embodiments, CaNCR peptide variant can be optimized for lower IC50 and/or MIC50 values against one or more microbial pathogens by selecting for CaNCR peptide variant that provide for improved phospholipid binding in comparison to a reference protein containing just one of the defensin peptides. Suitable assays for determining improved phospholipid include protein-lipid overlay assays (e.g., Dowler et al., 2002), surface plasmon resonance assays (e.g., Baron and Pauron, 2014), biotin capture lipid affinity assays (e.g., Davidson et al., 2006), titration calorimetry assays (e.g., Miller and Cistola, 1993), and the like.

Expression cassettes that provide for expression of the CaNCR peptide variant or CaNCR peptide variant multimer in monocotyledonous plants, dicotyledonous plants, or both can be constructed. Such CaNCR peptide variant or protein expression cassette construction can be effected either in a plant expression vector or in the genome of a plant. Expression cassettes are DNA constructs wherein various promoter, coding, and polyadenylation sequences are operably linked. In general, expression cassettes typically comprise a promoter that is operably linked to a sequence of interest, which is operably linked to a polyadenylation or terminator region. In certain instances including, but not limited to, the expression of recombinant or edited polynucleotides in monocot plants, it can also be useful to include an intron sequence. When an intron sequence is included, it is typically placed in the 5′ untranslated leader region of the recombinant or edited polynucleotide. In certain instances, it can also be useful to incorporate specific 5′ untranslated sequences in a recombinant or edited polynucleotide to enhance transcript stability or to promote efficient translation of the transcript.

A variety of promoters can be used to express the CaNCR peptide variants. One broad class of useful promoters are referred to as “constitutive” promoters in that they are active in most plant organs throughout plant development. For example, the promoter can be a viral promoter such as a CaMV35S or FMV35S promoter. The CaMV35S and FMV35S promoters are active in a variety of transformed plant tissues and most plant organs (e.g., callus, leaf, seed and root). Enhanced or duplicate versions of the CaMV35S and FMV35S promoters are particularly useful (U.S. Pat. No. 5,378,619, incorporated herein by reference in its entirety). Other useful promoters include the nopaline synthase (NOS) and octopine synthase (OCS) promoters (which are carried on tumor-inducing plasmids of A. tumefaciens), the cauliflower mosaic virus (CaMV) 19S promoters, a maize ubiquitin promoter, the rice Act1 promoter, and the Figwort Mosaic Virus (FMV) 35S promoter (see, e.g., U.S. Pat. No. 5,463,175, incorporated herein by reference in its entirety). It is understood that this group of exemplary promoters is non-limiting and that one skilled in the art could employ other promoters that are not explicitly cited here to express CaNCR peptide variants or CaNCR peptide variant multimers.

Promoters that are active in certain plant tissues (i.e., tissue specific promoters) can also be used to drive expression of CaNCR peptide variants. Expression of CaNCR peptide variants in the tissue that is typically infected by a microbial pathogen is anticipated to be particularly useful. Thus, expression in reproductive tissues, seeds, roots, stems, or leaves can be particularly useful in combating infection of those tissues by certain microbial pathogens in certain crops. Examples of useful tissue-specific, developmentally regulated promoters include but are not limited to the β-conglycinin 7S promoter (Doyle et al., 1986), seed-specific promoters (Lam and Chua, 1991), and promoters associated with napin, phaseolin, zein, soybean trypsin inhibitor, ACP, stearoyl-ACP desaturase, or oleosin genes. Examples of root specific promoters include but are not limited to the RB7 and RD2 promoters described in U.S. Pat. Nos. 5,459,252 and 5,837,876, respectively.

Another class of useful promoters are promoters that are induced by various environmental stimuli. Promoters that are induced by environmental stimuli include promoters induced by heat (e.g., heat shock promoters such as Hsp70), promoters induced by light (e.g., the light-inducible promoter from the small subunit of ribulose 1,5-bisphosphate carboxylase, ssRUBISCO, a very abundant plant protein), promoters induced by cold (e.g., COR promoters), promoters induced by oxidative stress (e.g., catalase promoters), promoters induced by drought (e.g., the wheat Em and rice rab16A promoters), and promoters induced by multiple environmental signals (e.g., rd29A promoters, Glutathione-S-transferase (GST) promoters).

Promoters that are induced by microbial infections in plants can also be used to drive expression of CaNCR peptide variants and proteins. Useful promoters induced by microbial infections include those promoters associated with genes involved in phenylpropanoid metabolism (e.g., phenylalanine ammonia lyase, chalcone synthase promoters), genes that modify plant cell walls (e.g., hydroxyproline-rich glycoprotein, glycine-rich protein, and peroxidase promoters), genes encoding enzymes that degrade microbial cell walls (e.g., chitinase or glucanase promoters), genes encoding thaumatin-like protein promoters, or genes encoding proteins of unknown function that display significant induction upon microbial infection. Maize and flax promoters, designated as Mis1 and Fis1, respectively, are also induced by microbial infections in plants and can be used (US Patent Appl. Pub. No. 20020115849).

Depending on the microbe to which protection is sought, the present CaNCR peptide variants can be expressed in any tissue or organ in the plant where the microbe attacks. In the case of Fusarium for example, a useful site for expression is in the roots. In the case of those microbes that infect by entering external plant surfaces, accumulation of the CaNCR peptide variants in the apoplast can be used. In certain embodiments, the apoplast-localized CaNCR peptide variant can be expressed in roots, stems, leaves, etc., by the use of tissue-specific promoters.

Promoters active at particular developmental stages in the plant life cycle can also be used to optimize resistance to microbial infection and/or damage when it is most needed.

An intron can also be included in the DNA expression construct, especially in instances when the sequence of interest is to be expressed in monocot plants. For monocot plant use, introns such as the maize hsp70 intron (U.S. Pat. No. 5,424,412; incorporated by reference herein in its entirety), the maize ubiquitin intron, the Adh intron 1 (Callis et al., 1987), the sucrose synthase intron (Vasil et al., 1989) or the rice Act1 intron (McElroy et al., 1990) can be used. Dicot plant introns that are useful include introns such as the CAT-1 intron (Cazzonnelli and Velten, 2003), the pKANNIBAL intron (Wesley et al., 2001; Collier et al., 2005), the PIV2 intron (Mankin et al., 1997) and the “Super Ubiquitin” intron (U.S. Pat. No. 6,596,925, incorporated herein by reference in its entirety; Collier et al., 2005) that have been operably integrated into recombinant or edited polynucleotides. It is understood that this group of exemplary introns is non-limiting and that one skilled in the art could employ other introns that are not explicitly cited here to express CaNCR peptide variants or CaNCR peptide variant multimers.

Certain embodiments comprise a sequence encoding an apoplast localization peptides that facilitates secretion of the mature CaNCR peptide variants from plant cells. Apoplast localization peptides include peptides referred to as signal peptides. In certain embodiments, apoplast localization peptides can be operably linked to the n-termini of CaNCR peptide variants to provide for apoplast localization. Portions of NCR, defensin, or other proproteins that encode apoplast localization peptides (e.g., signal peptides) can be used for secreting CaNCR peptide variants from plant or other cells. Examples of NCR proproteins that contain apoplast localization peptides that can be used in CaNCR peptide variants include the NCR proproteins of disclosed in Montiel et al. 2016 Molec. Plant Microb. Inter. 29:210-219. Examples of defensin proproteins that contain apoplast localization peptides that can be used in CaNCR peptide variants include the defensin proproteins of disclosed in U.S. Pat. No. 7,825,297 and US Patent Appl. Pub. No. 20160208278 (each incorporated herein by reference in their entireties), proteins that have at least about 70%, 80%, 90%, 95%, or 99% sequence identity to these sequences, and the biological functional equivalents of these sequences. Alternatively, signal peptide sequences derived from other Medicago defensin proteins (Hanks et al., 2005) can be used. Examples of such other Medicago defensin protein signal peptides include signal peptides of MtDef1.1 and MtDef2.1. Another example of a useful signal peptide encoding sequence that can be used in monocot plants is the signal peptide derived from a barley cysteine endoproteinase gene (Koehler and Ho, 1990) or an alpha-amylase gene. Another example of a useful signal peptide encoding sequence that can be used in dicot plants is the tobacco PR1b signal peptide. In other embodiments, wholly synthetic signal peptides can be used. This group of signal peptides is meant to be exemplary and non-limiting, and one skilled in the art could employ other signal peptides that are not explicitly cited here.

In other embodiments, sequences encoding peptides that provide for the localization of CaNCR peptide variants in subcellular organelles can be operably linked to the sequences that encode the CaNCR peptide variants. CaNCR peptide variants that are operably linked to a signal peptide are expected to enter the secretion pathway and can be retained by organelles such as the endoplasmic reticulum (ER) or targeted to the vacuole by operably linking the appropriate retention or targeting peptides to the C-terminus of the CaNCR peptide variant or protein. Examples of vacuolar targeting peptides include a CTPP vacuolar targeting signal from the barley lectin gene. Examples of ER targeting peptides include a peptide comprising a KDEL amino acid sequence.

In certain embodiments, a plastid localization peptide can be operably linked to the CaNCR peptide variants to provide for localization of the CaNCR peptide variants in a plant plastid. Plastid transit peptides can be obtained from nuclear-encoded and plastid localized proteins that include Rubisco small subunit (RbcS), chlorophyll a/b-binding protein, ADP-glucose pyrophosphorylase (ADPGPP), and the like. Plastid targeting peptides that been disclosed in non-patent (Li and Teng, 2013) and patent literature (US Patent Appl. Pub. No. 20160017351 and U.S. Pat. No. 5,510,471, each incorporated herein by reference in their entireties). Chimeric plastid targeting peptides have also been disclosed (Lee et al., Plant Physiol., 2015). Any of the aforementioned plastic targeting peptides can be adapted for use in localizing CaNCR peptide variants in plastids. In certain embodiments, the plastid localization peptide can be operably linked to the N-terminus of the CaNCR peptide variants.

In certain embodiments, a mitochondrial localization peptide can be operably linked to the CaNCR peptide variants to provide for localization of the CaNCR peptide variants in the mitochondria. Mitochondrial localization peptides can be obtained from nuclear-encoded and mitochondrial localized proteins that include beta-subunit of the F (1)-ATP synthase, alternative oxidases, and the gamma-subunit of the F (1)-ATP synthase. Mitochondrial targeting peptides have been disclosed (Sjoling and Glaser; 1998; Huang et al., Plant Physiology, 2009). In certain embodiments, the mitochondrial localization peptide will be operably linked to the N-terminus of the CaNCR peptide variants. Any of the aforementioned mitochondrial targeting peptides can be adapted for use in localizing CaNCR peptide variants or CaNCR peptide variant multimers in mitochondria. In certain embodiments, the mitochondrial localization peptide can be operably linked to the N-terminus of the CaNCR peptide variants.

In still other embodiments, dual localization peptide(s) can be used to provide for localization of the CaNCR peptide variants in both plastids and mitochondria (Carrie and Small, 2013).

Localization of CaNCR peptide variants or CaNCR peptide variant multimers in the apoplast, endoplasmic reticulum, the vacuole, plastids, or mitochondria can provide for useful properties such as increased expression in transgenic or edited plants and/or increased efficacy in inhibiting microbial growth in transgenic or edited plants. In certain embodiments, the localization peptide is a heterologous localization peptide that can direct an operably associated protein or peptide to an extracellular or sub-cellular location that is different than the extracellular or sub-cellular location of a naturally occurring protein or antimicrobial peptides. In certain embodiments, the localization peptide can target a CaNCR peptide variant multimer that comprises a spacer peptide, linker peptide, or junction sequence that is susceptible to cleavage by a plant endoproteinase to an extracellular or sub-cellular compartment where activity of that plant endoproteinase reduced or absent and thus provide for accumulation of the CaNCR peptide variant multimer in the transgenic or edited plant.

In other embodiments, the CaNCR peptide variant sequence-, defensin-, localization-, spacer-, or other peptide or protein encoding nucleotide sequence can be synthesized de novo from a CaNCR peptide variant sequence-, defensin-, localization-, spacer-, or other peptide or protein disclosed herein. The sequence of the peptide or protein-encoding nucleotide sequence can be deduced from the CaNCR peptide variant-, defensin-, localization-, spacer-, or other protein sequence through use of the genetic code. Computer programs such as “BackTranslate” (GCG™ Package, Acclerys, Inc. San Diego, CA) can be used to convert a peptide sequence to the corresponding nucleotide sequence that encodes the peptide.

Furthermore, the synthetic CaNCR peptide variant-, defensin-, localization-, spacer-, or other peptide or protein nucleotide sequence can be designed so that it will be optimally expressed in plants. U.S. Pat. No. 5,500,365 describes a method for synthesizing plant genes to optimize the expression level of the protein encoded by the synthesized gene. This method relates to the modification of the structural gene sequences of the exogenous recombinant or edited polynucleotide, to make them more “plant-like” and therefore more efficiently transcribed, processed, translated, and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052.

In certain embodiments, a CaNCR peptide variant encoding sequence can also be operably linked to a 3′ non-translated region containing a polyadenylation signal. This polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene 3′ un-translated regions contain polyadenylate signals and represent non-limiting examples of such 3′ untranslated regions that can be used. It is understood that this group of polyadenylation regions is non-limiting and that one skilled in the art could employ other polyadenylation regions that are not explicitly cited here.

The DNA constructs that comprise the plant expression cassettes described above can either be constructed in the plant genome by using site-specific insertion of heterologous DNA into the plant genome, by mutagenizing the plant genome, and/or by introducing the expression cassette into the plant genome with a vector or other DNA transfer method. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in both E. coli and Agrobacterium as well as one or more “border” sequences positioned so as to permit integration of the expression cassette into the plant chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts.

Methods of obtaining a transgenic or edited plant capable of inhibiting growth of a plant pathogenic microbe are also provided. In one embodiment, expression vectors suitable for expression of the CaNCR peptide variant in various dicot and monocot plants are introduced into a plant, a plant cell, a protoplast, or a plant tissue using transformation techniques as described herein. In another embodiment, the CaNCR peptide variant expression cassette is constructed in the plant nuclear or plastid genome by editing. Next, a transgenic or edited plant containing or comprising the CaNCR peptide variant expression vector is obtained by regenerating that transgenic or edited plant from the plant, plant cell, protoplast, or plant tissue that received the expression vector or genome edits. The final step is to obtain a transgenic or edited plant that expresses a plant pathogenic microbe inhibitory amount of the mature CaNCR peptide variant, where a “plant pathogenic microbe inhibitory amount” is a level of CaNCR peptide variant sufficient to provide any measurable decrease in microbial growth in the transgenic or edited plant and/or any measurable decrease in the adverse effects caused by microbial growth in the transgenic or edited plant.

Any of the CaNCR peptide variant expression vectors can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. The aforementioned methods of introducing transgenes are described in US Patent Appl. Pub. No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., 2005. It is further understood that the CaNCR peptide variant expression vector can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the expression vector into the chromosome of a plant cell, a protoplast, a plant tissue, or a plant.

Methods of introducing plant mini-chromosomes comprising plant centromeres that provide for the maintenance of the recombinant mini-chromosome in a transgenic plant (U.S. Pat. Nos. 6,972,197 and 8,435,783) can also be used to introduce and maintain CaNCR peptide variant in such plants. In these embodiments, the transgenic plants harbor the mini-chromosomes as extrachromosomal elements that are not integrated into the chromosomes of the host plant.

In certain embodiments, transgenic plants can be obtained by linking the gene of interest (in this case an CaNCR peptide variant-encoding polynucleotide sequence) to a selectable marker gene, introducing the linked polynucleotides into a plant cell, a protoplast, a plant tissue, or a plant by any one of the methods described above, and regenerating or otherwise recovering the transgenic plant under conditions requiring expression of the selectable marker gene for plant growth. The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, or an aminoethylcysteine insensitive octopine synthase protein. The corresponding selective agents used in conjunction with each gene can be: neomycin (for neomycin phosphotransferase protein selection), phosphinothricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

In certain embodiments, a plant comprising a recombinant or edited polynucleotide encoding a CaNCR peptide variant or CaNCR peptide variant multimer can be obtained by using techniques that provide for site specific insertion of heterologous DNA into the genome of a plant (e.g., by editing). In certain embodiments, ta DNA fragment comprising at least one of an CaNCR peptide variant, a CaNCR peptide variant multimer, defensin peptide, a spacer peptide that is resistant to cleavage by a plant endoproteinase, a heterologous promoter, or a heterologous localization peptide, is site specifically integrated into the genome to a plant cell, tissue, part, or whole plant to create a sequence within that genome that encodes a CaNCR peptide variant or a CaNCR peptide variant multimer. In one embodiment of the method, the heterologous DNA encodes a spacer peptide sequence and a CaNCR peptide variant or defensin peptide that is inserted in-frame at either the N-terminus of an endogenous peptide coding region or at the C-terminus of an endogenous coding region to provide a transgenic or gene edited plant comprising genomic DNA encoding an endogenous peptide that is operably linked to a heterologous spacer peptide encoding DNA sequence and an CaNCR peptide variant. In certain embodiments, a heterologous promoter or promoter element can be inserted at or near the 5′ end of a genomic region that comprises a sequence encoding an CaNCR peptide variant or protein to obtain a transgenic or gene edited plant where the genomic region is under the transcriptional control of the inserted or composite promoter. In practicing any of the aforementioned methods, such heterologous DNA can either be inserted in a parallel (e.g., at the same time) or sequentially (e.g., at the distinct times). In one non-limiting example, a heterologous DNA encoding a CaNCR peptide variant can be inserted into an endogenous genomic region encoding an endogenous peptide at the same time that a heterologous promoter, promoter element, and/or localization peptide is inserted into the genomic region. Examples of methods for inserting foreign DNA at specific sites in the plant genome with site-specific nucleases such as meganucleases or zinc-finger nucleases are at least disclosed in Voytas, 2013. Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Murovec et al., 2017; Kumar and Jain, 2015; and in US Patent Appl. Pub. No. 20150082478, which is specifically incorporated herein by reference in its entirety.

In certain embodiments, a genetically edited plant comprising a recombinant or edited polynucleotide encoding a CaNCR peptide variant or CaNCR peptide variant multimer can be obtained by using techniques that provide for genome editing in the plant. Examples of methods for plant genome editing with clustered regularly interspaced short palindromic repeats (CRISPR)-associated (Cas)-polynucleotide modification template technology and a Cas endonuclease are at least disclosed by Svitashev et al., 2015; Kumar and Jain, 2015; Murovec et al., 2017; and in US Patent Appl. Pub. No. 20150082478, which is specifically incorporated herein by reference in its entirety. Examples of additional methods for editing plant genomes through use of Cpf1 or Csm1 nucleases are disclosed in US Patent Application Publication 20180148735, which is incorporated herein by reference in its entirety.

Transgenic plants can also be obtained by linking a gene of interest (in this case a CaNCR peptide variant-encoding polynucleotide sequence) to a scoreable marker gene, introducing the linked polynucleotides into a plant cell by any of the methods described above, and regenerating the transgenic plants from transformed plant cells that test positive for expression of the scoreable marker gene. The scoreable marker gene can be a gene encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein, or a chloramphenicol acetyl transferase protein.

When an expression vector encoding an CaNCR peptide variant is introduced into a plant cell or plant tissue or when an CaNCR peptide variant is introduced in the genome of a plant cell or tissue by site specific insertion of heterologous DNA into the plant genome, the transformed cells or tissues can be regenerated into whole plants by culturing these cells or tissues under conditions that promote the formation of a whole plant (i.e., the process of regenerating leaves, stems, roots, and, in certain plants, reproductive tissues). The development or regeneration of transgenic plants from either single plant protoplasts or various explants has been described (Horsch, R. B. et al., 1985). This regeneration and growth process typically includes the steps of selection of transformed cells and culturing selected cells under conditions that will yield rooted plantlets. The resulting transgenic rooted shoots are thereafter planted in an appropriate plant growth medium such as soil. Alternatively, transgenes can also be introduced into isolated plant shoot meristems and plants regenerated without going through callus stage tissue culture (U.S. Pat. No. 7,002,058). When the transgene is introduced directly into a plant, or more specifically into the meristematic tissue of a plant, seed can be harvested from the plant and selected or scored for presence of the transgene. In the case of transgenic plant species that reproduce sexually, seeds can be collected from plants that have been “selfed” (self-pollinated) or out-crossed (i.e., used as a pollen donor or recipient) to establish and maintain the transgenic plant line. Transgenic plants that do not sexually reproduce can be vegetatively propagated to establish and maintain the transgenic plant line. In certain embodiments, transgenic plants are derived from a transformation event where the transgene has inserted into one or more locations in the plant genome. In certain embodiments, a seed produced by the transgenic plant, a progeny from such seed, and a seed produced by the progeny of the original transgenic plant are provided. Such progeny and seeds will have a CaNCR peptide variant or CaNCR peptide variant multimer-encoding recombinant or edited polynucleotide stably incorporated into their genome, and such progeny plants will inherit the traits afforded by the introduction of a stable recombinant or edited polynucleotide in Mendelian fashion. It is further recognized that transgenic or edited plants containing the CaNCR peptide variant encoding DNA constructs or edits described herein, and materials derived therefrom, can be identified through use of PCR or other methods that can specifically detect the sequences in the DNA constructs. Methods developed for regeneration and propagation of transgenic plants can be adapted for regeneration and propagation of edited plants.

Once a transgenic or edited plant is regenerated or recovered, a variety of methods can be used to identify or obtain a transgenic or edited plant that expresses a plant pathogenic microbe inhibitory amount of CaNCR peptide variant. One general set of methods is to perform assays that measure the amount of CaNCR peptide variant that is produced. For example, various antibody-based detection methods employing antibodies that recognize CaNCR peptide variant can be used to quantitate the amount of CaNCR peptide variant produced. Examples of such antibody-based assays include ELISAs, RIAs, or other methods wherein an CaNCR peptide variant-recognizing antibody is detectably labelled with an enzyme, an isotope, a fluorophore, a lanthanide, and the like. By using purified or isolated CaNCR peptide variant as a reference standard in such assays (i.e., providing known amounts of CaNCR peptide variant), the amount of CaNCR peptide variant present in the plant tissue in a mole per gram of plant material or mass per gram of plant material can be determined. The CaNCR peptide variant will typically be expressed in the transgenic or edited plant at the level of “parts per million” or “PPM”, where microgram levels of CaNCR peptide variant are present in gram amounts of fresh weight plant tissue. In this case, 1 microgram of CaNCR peptide variant per 1 gram of fresh weight plant tissue would represent a CaNCR peptide variant concentration of 1 PPM. A plant pathogenic microbe inhibitory amount of CaNCR peptide variant is at least about 0.05 PPM (i.e., 0.05 μg CaNCR peptide variant per gram fresh weight plant tissue) or at least about 0.1 PPM. In certain embodiments, a plant pathogenic microbe inhibitory amount of CaNCR peptide variant is at least about 0.5 PPM. In certain embodiments, the amount of CaNCR peptide variant is at least about 1.0 PPM. In certain embodiments, the amount of CaNCR peptide variant is at least about 2.0 PPM. In certain embodiments, the amount of the CaNCR peptide variant multimer is at least about 0.05 PPM, 0.1 PPM, 0.5 PPM, or 1.0 PPM to about 5, 10, 20, 50, 100, 200, 500, or 1000 PPM. In certain embodiments, including those where a plastid genome is transformed or edited to express a CaNCR peptide variant or CaNCR peptide variant multimer, about 0.1%, 0.2% or 0.5% to about 1%, 3%, 5%, or more of the soluble protein in a plant part, including a leaf, can be the CaNCR peptide variant or CaNCR peptide variant multimer.

Alternatively, the amount of CaNCR peptide variant-encoding mRNA produced by the transgenic or edited plant can be determined to identify plants that express plant pathogenic microbe inhibitory amounts of CaNCR peptide variant. Techniques for relating the amount of protein produced to the amount of RNA produced include methods such as constructing a standard curve that relates specific RNA levels (i.e., CaNCR peptide variant mRNA) to levels of the CaNCR peptide variant (determined by immunologic or other methods). Methods of quantitating CaNCR peptide variant mRNA typically involve specific hybridization of a polynucleotide to either the CaNCR peptide variant mRNA or to a cDNA (complementary DNA) or PCR product derived from the CaNCR peptide variant RNA. Such polynucleotide probes can be derived from either the sense and/or antisense strand nucleotide sequences of the CaNCR peptide variant-encoding recombinant or edited polynucleotide. Hybridization of a polynucleotide probe to the CaNCR peptide variant mRNA or cDNA can be detected by methods including, but not limited to, use of probes labelled with an isotope, a fluorophore, a lanthanide, or a hapten such as biotin or digoxigenin. Hybridization of the labelled probe can be detected when the CaNCR peptide variant RNA is in solution or immobilized on a solid support such as a membrane. When quantitating CaNCR peptide variant RNA by use of a quantitative reverse-transcriptase Polymerase Chain Reaction (qRT-PCR), the PCR product can be detected by use of any of the aforementioned labelled polynucleotide probes, by use of an intercalating dye such as ethidium bromide or SYBR green, or use of a hybridization probe containing a fluorophore and a quencher such that emission from the fluorophore is only detected when the fluorophore is released by the 5′ nuclease activity of the polymerase used in the PCR reaction (i.e., a TaqMan™ reaction; Applied Biosystems, Foster City, CA) or when the fluorophore and quencher are displaced by polymerase mediated synthesis of the complementary strand (i.e., Scorpion™ or Molecular Beacon™ probes). Various methods for conducting qRT-PCR analysis to quantitate mRNA levels are well characterized (Bustin, S. A.; 2002). Fluorescent probes that are activated by the action of enzymes that recognize mismatched nucleic acid complexes (i.e., Invader™, Third Wave Technologies, Madison, WI) can also be used to quantitate RNA. Those skilled in the art will also understand that RNA quantitation techniques such as Quantitative Nucleic Acid Sequence Based Amplification (Q-NASBA™) can be used to quantitate CaNCR peptide variant-encoding mRNA and identify expressing plants.

Transgenic or edited plants that express plant pathogenic microbe inhibitory amounts of CaNCR peptide variants can also be identified by directly assaying such plants for inhibition of the growth of a plant pathogenic microbe. Such assays can be used either independently or in conjunction with MDD expression assays to identify the resistant transgenic or edited plants.

Infection of certain plants with certain plant pathogen microbes can result in distinctive effects on plant growth that are readily observed. Consequently, one can distinguish CaNCR peptide variant-expressing transgenic or edited plants by simply challenging such plants transformed with CaNCR peptide variant-encoding recombinant or edited polynucleotides with pathogenic plant microbes and observing reduction of the symptoms normally associated with such infections. Such observations are facilitated by co-infecting otherwise identical, non-transgenic control plants that do not contain a CaNCR peptide variant encoding recombinant or edited polynucleotide with the same type and dose of plant pathogenic microbes used to infect the transgenic or edited plants that contain an CaNCR peptide variant-encoding recombinant or edited polynucleotide. Identification of transgenic or edited plants that control or combat microbial infection can be based on observation of decreased disease symptoms, measurement of the decreased microbial growth in the infected plant (e.g., by determining the numbers of colony forming units per gram of infected tissue) and/or by measurement of the amount of mycotoxins present in infected plant tissue. The use of microbial disease severity assays and colony formation assays in conjunction with expression assays to identify transgenic MsDef1-expressing potato plants that are resistant to Verticillium dahliae has been described (U.S. Pat. No. 6,916,970 and Gao et. al., 2000). It is similarly anticipated that a variety of CaNCR peptide variant-expressing transgenic or edited plants that combat or control microbial pathogens can be identified by scoring transgenic or edited plants for resistance to microbial pathogens that infect those plants. Examples of CaNCR peptide variant recombinant or edited polynucleotide-conferred microbial resistance that can be assayed by observing reductions in disease symptoms or reductions in microbial growth include resistance of transgenic or edited corn to Fusarium verticillioides, Fusarium moniliforme, Colletotrichum graminicola, Stenocarpella maydis, and/or Cercospora zeae-maydis; resistance of transgenic or edited wheat to head blight (Fusarium graminearum), powdery mildew (Erysiphe graminis f. sp. tritici), stripe rust, stem rust or leaf rust (Puccinia tritici); resistance of transgenic or edited cotton to Fusarium oxysporum and Verticillium dahlia; resistance of transgenic or edited rice to Magnaporthe oryzae and Rhizoctonia solani, and resistance of transgenic or edited soybean to Asian Soybean rust (Phakopsora pachyrhizi), Phytophthora Root Rot (Phytophthora sp.), White Mold (Sclerotinia sp.), Sudden Death Syndrome (Fusarium virguliforme) and/or Brown Stem Rot (Phialophora gregata).

Transgenic or edited plants that express plant pathogenic microbe inhibitory amounts of CaNCR peptide variant can also be identified by measuring decreases in the adverse effects cause by microbial growth in such plants. Such decreases can be ascertained by comparing the extent of the adverse effect in a CaNCR peptide variant-expressing transgenic or edited plant relative to an otherwise identical, non-transgenic or unedited control plant that does not express an CaNCR peptide variant. Adverse effects of microbial growth in a plant that can be measured include any type of plant tissue damage or necrosis, any type of plant yield reduction, any reduction in the value of the crop plant product, and/or production of undesirable microbial metabolites or microbial growth by-products including, but not limited to, mycotoxins. Mycotoxins comprise a number of toxic molecules produced by microbial species, including but not limited to polyketides (including aflatoxins, demethylsterigmatocystin, O-methylsterigmatocystin, etc.), fumonisins, alperisins (e.g., Als A2, Bls B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

A wide variety of plants that express CaNCR peptide variant can either be constructed by using site specific insertion of heterologous DNA into the plant genome, by mutagenizing the plant genome, and/or by introducing the expression cassette into the plant genome with a vector or other DNA transfer method to obtain transgenic or edited plants that combat or control microbial infections, or that resist such infections.

Plants of interest include both food crop plants and biofuels or energy crop plants, as listed above. Transgenic or edited monocot plants obtainable by the expression vectors and methods described herein include but are not limited to barley, corn, flax, oat, rice, rye, sorghum, turf grass, sugarcane, and wheat. Transgenic or edited dicot plants obtainable by the expression vectors and methods described herein include but are not limited to alfalfa, Arabidopsis, barrel medic, banana, broccoli, bean, cabbage, canola, carrot, cassava, cauliflower, celery, citrus, cotton, cucurbits, eucalyptus, garlic, grape, onion, lettuce, pea, peanut, pepper, potato, poplar, pine, sunflower, safflower, soybean, strawberry, sugar beet, sweet potato, tobacco, and tomato.

Expression of CaNCR peptide variants in yeast is also specifically contemplated herein. The construction of expression vectors for production of heterologous proteins in various yeast genera is well established. In general, such expression vectors typically comprise a promoter that is operably linked to a sequence of interest, which is operably linked to a polyadenylation or terminator region. Examples of yeast genera that have been used to express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985). Expression vectors and transformation systems useful for yeasts other than Saccharomyces are described in Reiser et al (1990).

In general, the promoter and polyadenylation region are selected based on their operability in a given yeast host. For example, the AOX1 or AOX2 promoters of Pichia can be used in conjunction with the AOX1, AOX2, p40, or p76 polyadenylation sequences of Pichia to express a heterologous protein such as a CaNCR peptide variant. Both the AOX1 and AOX2 promoters are particularly useful in Pichia as both promoters provide for abundant expression of the linked heterologous gene when induced by addition of methanol to the growth medium. The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous proteins such as CaNCR peptide variant in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as CaNCR peptide variant-encoding genes. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast expression vectors that provide for secretion of heterologous proteins such as CaNCR peptide variants or CaNCR peptide variant multimers into the growth medium by transformed yeast are also contemplated. Secretion of the CaNCR peptide variant or CaNCR peptide variant multimer is typically achieved by operable linkage of a signal peptide sequence or a signal peptide and propeptide sequence to the CaNCR peptide variant or CaNCR peptide variant multimer encoding sequence. Examples of useful signal peptides for secretion of heterologous proteins in yeast include but are not limited to an alpha-factor signal peptide, an invertase signal peptide, and a PHO1 signal peptide, all of which are derived from yeast. The alpha-factor signal peptide is typically derived from Saccharomyces, Kluveromyces, or Candida, while the PHO1 signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is expressly incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the CaNCR peptide variant or CaNCR peptide variant multimer to provide for secretion of the CaNCR peptide variant or CaNCR peptide variant multimer. In this case, the signal peptide is cleaved from the CaNCR peptide variant or CaNCR peptide variant multimer in the course of the secretion process. Alternatively, the signal peptide and propeptide of the alpha mating factor can be operably linked to the CaNCR peptide variant or CaNCR peptide variant multimer via a cleavage site sequence. This cleavage site sequence can comprise a variety of sequences that provide for proteolytic processing of the leader sequence and gene of interest. In the native S. cerevisiae alpha mating factor gene the cleavage site sequence corresponds to amino acid residues 84-89 and is represented by the sequence Lys84-Arg85-Glu86-Ala87-Glu88-Ala 89. The sequence Lys-Arg corresponds to a KEX2 protease recognition site while the Glu-Ala-Glu-Ala sequence corresponds to a duplicated dipeptidylaminopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylaminopeptidase or STE13 recognition site at residues 86-89) is operably linked to the sequence encoding the mature CaNCR peptide variants or CaNCR peptide variant multimer. When the N-terminal 89 amino acids of the alpha mating factor precursor protein are fused to the N-terminus of a heterologous protein such as a CaNCR peptide variant or CaNCR peptide variant multimer, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature CaNCR peptide variants or CaNCR peptide variant multimer. When the N-terminal 85 amino acids of the alpha mating factor precursor protein are fused to the N-terminus of a heterologous protein such as CaNCR peptide variant or CaNCR peptide variant multimer, the propeptide sequence is typically dissociated from the heterologous protein via cleavage by endogenous yeast proteases at the KEX2 recognition site. The CaNCR peptide variants or CaNCR peptide variant multimer can thus be expressed without the glu-ala repeats.

To obtain transformed yeast that express CaNCR peptide variant or CaNCR peptide variant multimer, the yeast expression cassettes (e.g., yeast promoter, yeast signal peptide encoding sequence, mature CaNCR peptide variants or CaNCR peptide variant multimer sequence, and polyadenylation sequence) are typically combined with other sequences that provide for selection of transformed yeast. Examples of useful selectable marker genes include genes encoding a ADE protein, a HIS5 protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a AR04-OFP protein, and a FZF1-4 protein.

DNA molecules comprising the yeast CaNCR peptide variant expression cassettes and selectable marker genes are introduced into yeast cells by techniques such as transfection into yeast spheroplasts or electroporation. In certain embodiments, the DNA molecules comprising the yeast CaNCR peptide variant expression cassettes and selectable marker genes are introduced as linear DNA fragments that are integrated into the genome of the transformed yeast host cell. Integration can occur at random sites in the yeast host cell genome or at specific sites in the yeast host cell genome. Integration at specific sites in the yeast host cell genome is typically accomplished by homologous recombination between sequences contained in the expression vector and sequences in the yeast host cell genome. Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Yeast cells transformed with the yeast CaNCR peptide variant or CaNCR peptide variant multimer expression cassettes can be used to produce CaNCR peptide variant or CaNCR peptide variant multimer. In certain embodiments, the CaNCR peptide variant or CaNCR peptide variant multimer molecules can be used directly as antimicrobial agents, to produce antimicrobial compositions that can be applied to plants, processed plant products, animals, or human subjects, as immunogens to raise antibodies that recognize the CaNCR peptide variants, or as reference standards in kits for measuring concentrations of CaNCR peptide variants in various samples. The transformed yeast cells expressing CaNCR peptide variant or CaNCR peptide variant multimer antimicrobial molecules can also be applied to plants, processed plant products, animals, or humans to combat/control pathogenic microbial infections. The methods of producing CaNCR peptide variants typically first comprise the step of culturing yeast cells transformed antimicrobial peptides with CaNCR peptide variant or CaNCR peptide variant multimer expression cassettes under conditions wherein the yeast cells express a mature CaNCR peptide variant or CaNCR peptide variant multimer molecule. In general, the conditions where the yeast cells express the mature CaNCR peptide variant or CaNCR peptide variant multimer molecules are conditions that allow for or specifically induce expression of the yeast promoter that is operably linked to the CaNCR peptide variant coding sequence in the yeast expression cassette. When the yeast is Pichia and the signal-peptide/MD gene is under the control of an AOX1 or AOX2 promoter, addition of methanol to the growth medium will provide for expression of mature CaNCR peptide variants or CaNCR peptide variant multimer. Similarly, when the yeast is Hansuela and the signal-peptide/MD gene is under the control of a MOX, DHAS, or FMDH promoter, addition of methanol to the growth medium will provide for expression of mature CaNCR peptide variants or CaNCR peptide variant multimers.

Alternatively, when the yeast is Kluveromyces and the signal-peptide/De/5 gene is under the control of a Lactase promoter, addition of galactose to the growth medium will provide for expression of mature CaNCR peptide variants or CaNCR peptide variant multimers.

Once the transformed yeast culture has been incubated under culture conditions that provide for expression of mature CaNCR peptide variant or CaNCR peptide variant multimer for a sufficient period of time, the mature CaNCR peptide variant or CaNCR peptide variant multimer can be isolated from the culture. A sufficient time period can be determined by periodically harvesting portions or aliquots of the culture and assaying for the presence of CaNCR peptide variant. Analytical assays such as SDS-PAGE with protein staining, Western blot analysis, or any immunodetection method (e.g., such as an ELISA) can be used to monitor CaNCR peptide variant or CaNCR peptide variant multimer production. For example, incubation in the presence of methanol for between 1 to 8 days is sufficient to provide for expression of mature CaNCR peptide variant or CaNCR peptide variant multimer protein from the AOX1 promoter in Pichia.

Isolation of the CaNCR peptide variant from the culture can be partial or complete. For CaNCR peptide variant or CaNCR peptide variant multimer expression vectors where a yeast signal peptide is operably linked to the sequence encoding the mature CaNCR peptide variant or CaNCR peptide variant multimer, the mature CaNCR peptide variant or CaNCR peptide variant multimer can be recovered from the yeast cell culture medium. Yeast cell culture medium that contains the mature CaNCR peptide variant or CaNCR peptide variant multimer can be separated from the yeast cells by centrifugation or filtration, thus effecting isolation of mature CaNCR peptide variant or CaNCR peptide variant multimer. Yeast cell culture medium that contains the mature CaNCR peptide variant or CaNCR peptide variant multimer can be further processed by any combination of dialysis and/or concentration techniques (e.g., precipitation, lyophilization, filtration) to produce a composition containing the CaNCR peptide variant or CaNCR peptide variant multimer. Production of CaNCR peptide variant or CaNCR peptide variant multimer can also comprise additional purification steps that result in either a partially or completely pure preparation of the CaNCR peptide variant or CaNCR peptide variant multimer. To effect such purification, filtration size-exclusion membranes can be used. Alternatively, various types of chromatographic techniques such as size exclusion chromatography, ion-exchange chromatography, or affinity chromatography can be used to produce a partially or completely pure preparation of the CaNCR peptide variant or CaNCR peptide variant multimer.

Combinations of various isolation techniques can also be employed to produce the mature CaNCR peptide variant or protein. For example, the cell culture medium can be separated from the cells by centrifugation and dialyzed or adjusted. In certain embodiments, a buffer for dialysis or adjustment is a 25 mM sodium acetate buffer at about pH4.5-pH6.0. This dialysate is then subjected to ion-exchange chromatography. For example, a cation-exchange resin such as CM-Sephadex C-25 equilibrated with a 25 mM sodium acetate buffer at about pH6.0 can be used. The CaNCR peptide variant or CaNCR peptide variant multimer bound to the cation exchange resin is washed and then eluted. For example, the aforementioned column is washed with 25 mM sodium acetate buffer at about pH6.0 and subsequently eluted in 1M NaCl, 50 mM Tris, pH7.6. Fractions containing the CaNCR peptide variant or CaNCR peptide variant multimer are identified by an assay or by UV absorbance and then concentrated by a size-cutoff filtration membrane. The concentrated CaNCR peptide variant or CaNCR peptide variant multimer is then dialyzed to obtain an essentially or substantially pure CaNCR peptide variant or CaNCR peptide variant multimer in a buffer. Buffers include buffers such as 10 mM Tris, pH 7.6.

Also provided are antimicrobial compositions for agricultural, pharmaceutical, or veterinary use comprising an antimicrobial plant, antimicrobial human or veterinary, pathogenic microbe inhibitory amount (“antimicrobial effective amount”) of one or more the present isolated, purified antimicrobial CaNCR peptide variants, or biologically functional equivalents thereof. Such compositions can comprise one, or any combination of, CaNCR peptide variants disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, CaNCR peptide variant-susceptible pathogenic microbes associated with plant, human or animal microbial infections. Such antimicrobial compositions can be formulated for topical administration, and applied topically to either plants, the plant environment (including soil), or humans or animals.

Agricultural compositions comprising any of the present CaNCR peptide variant molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present CaNCR peptide variants with other pesticidally active substances, fertilizers, and/or growth regulators, etc., in the form of finished formulations or tank mixes.

Whether alone or in combination with other active agents, the present antimicrobial CaNCR peptide variants can be applied at a concentration in the range of from about 0.1 μg ml to about 100 mg ml, or from about 5 μg ml to about 5 mg ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added.

Numerous conventional microbial antibiotics and chemical fungicides with which the present CaNCR peptide variants can be combined are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. In addition, azole, triazole, and/or echinocandin fungicides can also be used. Other active ingredients that can be formulated in combination with the present antimicrobial peptides include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial CaNCR peptide variants can be formulated.

Agriculturally useful antimicrobial compositions encompassed herein also include those in the form of host cells, such as bacterial and microbial cells, capable of the producing the CaNCR peptide variants and proteins, and which can colonize plants, including roots, shoots, leaves, or other parts of plants. The term “plant-colonizing microorganism” is used herein to refer to a microorganism that is capable of colonizing the any part of the plant itself and/or the plant environment, including, and which can express the present CaNCR peptide variant antimicrobial peptides in the plant and/or the plant environment. A plant-colonizing microorganism is one that can exist in symbiotic or non-detrimental relationship with a plant in the plant environment. U.S. Pat. No. 5,229,112, which is incorporated herein by reference in its entirety, discloses a variety of plant-colonizing microorganisms that can be engineered to express antimicrobial proteins, and methods of use thereof, applicable to the CaNCR peptide variant antimicrobial peptides disclosed herein. Plant-colonizing microorganisms expressing the presently disclosed CaNCR peptide variant antimicrobial peptides useful in inhibiting microbial growth in plants include bacteria selected from the group consisting of Bacillus spp. including but not limited to Bacillus thuringiensis, Bacillus israelensis, and Bacillus subtilis, Candidatus Liberibacter asiaticus; Pseudomonas spp.; Arthrobacter spp., Azospyrillum spp., Clavibacter spp., Escherichia spp.; Agrobacterium spp., for example A. radiobacter, Rhizobium spp., Erwinia spp. Azotobacter spp., Azospirillum spp., Klebsiella spp., Alcaligenes spp., Rhizobacterium spp., Xanthomonas spp., Ralstonia spp. and Flavobacterium spp., In certain embodiments, the microorganism is a yeast selected from the group consisting of Saccharomyces cerevisiae, Pichia pastoris, and Pichia methanolica. In certain embodiments, the plant-colonizing microorganism can be an endophytic bacteria or microbe.

When applying the present CaNCR peptide variant molecules to the rhizosphere, rhizosphere-colonizing bacteria from the genus Pseudomonas are particularly useful, especially the fluorescent pseudomonads, e.g., Pseudomonas fluorescens, which is especially competitive in the plant rhizosphere and in colonizing the surface of the plant roots in large numbers. Examples of suitable phylloplane (leaf) colonizing bacteria are P. putida, P. syringae, and Erwinia species.

The antimicrobial plant-colonizing microorganisms that can express CaNCR peptide variant can be applied directly to the plant, e.g., to the surface of leaves, buds, roots, shoots, floral parts, seeds, etc., or to the soil. When used as a seed coating, the plant-colonizing microorganisms that can express CaNCR peptide variant are applied to the plant seed prior to planting. The determination of an antimicrobial effective amount of plant-colonizing microorganisms used for a particular plant can be empirically determined and will depend on such factors as the plant species, the microbial pathogen, method of planting, and the soil type, (e.g., pH, organic matter content, moisture content). At least one, 10 or 100 plant-colonizing microorganism(s) containing DNA encoding the CaNCR peptide variant antimicrobial peptides disclosed herein is sufficient to control microbial pathogens because it or they can grow into a colony of clones of sufficient number to express antimicrobial amounts of the CaNCR peptide variant. However, in practice, due to varying environmental factors which can affect the survival and propagation of the microorganism, a sufficient number of plant colonizing microorganisms should be provided in the seed, plant or plant environment (e.g., roots or foliage) to assure survival and/or proliferation. For example, application of 103 to 1010 bacteria or yeasts per seed can be sufficient to insure colonization on the surface of the roots by the microorganism. In certain embodiments, it is sufficient to dose the plant or plant environment with enough bacteria or other plant-colonizing microorganism to maintain a population that expresses 100 to 250 nanograms of the CaNCR peptide variant per plant. For example, 105 to 108 bacteria per square centimeter of plant surface can be adequate to control microbial infection. In certain embodiments, at least about 5 or 10 nanograms to about 100, 200, 500, or 1,000 nanograms, of a CaNCR peptide variant or CaNCR peptide variant multimer can be sufficient to control microbial damage to plants.

Compositions containing the plant colonizing microorganisms that express the CaNCR peptide variant can be prepared by formulating the biologically active microorganism with adjuvants, diluents, carriers, etc., to provide compositions in the form of finely-divided particulate solids, granules, pellets, wettable powders, dusts, aqueous suspensions, dispersions, or emulsions. Illustrative of suitable carrier vehicles are solvents, e.g., water or organic solvents, and finely divided solids, e.g., kaolin, chalk, calcium carbonate, talc, silicates, and gypsum. In certain embodiments, plant-colonizing microorganisms that express the CaNCR peptide variant can also be in encapsulated form, e.g., the plant-colonizing microorganisms can be encapsulated within shell walls of polymer, gelatin, lipid, and the like. Other formulation aids such as, for example, emulsifiers, dispersants, surfactants, wetting agents, anti-foam agents, and anti-freeze agents, can be incorporated into the antimicrobial compositions, especially if such compositions will be stored for any period of time prior to use.

In addition to the plant-colonizing microorganisms that express CaNCR peptide variant, the compositions provided herein can additionally contain other known biologically active agents, such as, for example, a fungicide, herbicide, or insecticide. In addition, two or more plant-colonizing microorganisms that express either a different or the same CaNCR peptide variant can be combined.

The application of antimicrobial compositions containing the genetically engineered plant-colonizing microorganisms that can express CaNCR peptide variant as the active agent can be carried out by conventional techniques utilizing, for example, spreaders, power dusters, boom and hand sprayers, spry dusters, and granular applicators.

The compositions provided herein can be applied in an antimicrobial effective amount, which will vary depending on such factors as, for example, the specific fungal pathogen to be controlled, the specific plant (and plant part or soil) to be treated, and the method of applying the compositions that comprise CaNCR peptide variants and proteins.

CaNCR peptide variants and biologically functional equivalents, as well as transgenic or genetically edited plants or microorganisms expressing those proteins, can be used to inhibit the growth of a wide variety of susceptible microbes in plants. In certain embodiments, growth of microbes in the following genera or species can be inhibited: Alternaria (e.g., Alternaria brassicicola; Alternaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia maydis); Erysiphe (e.g., Erysiphe graminis f.sp. graminis; Erysiphe graminis f.sp. hordei); Fusarium (e.g., Fusarium nivale; Fusarium oxysporum; Fusarium graminearum; Fusarium culmorum; Fusarium solani; Fusarium moniliforme; Fusarium roseum); Gaeumanomyces (e.g., Gaeumanomyces graminis f.sp. tritici); Helminthosporium (e.g., Helminthosporium turcicum; Helminthosporium carbonum; Helminthosporium maydis); Macrophomina (e.g., Macrophomina phaseolina; Magnaporthe grisea); Nectria (e.g., Nectria heamatococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f.sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Sclerotium (e.g., Sclerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium albo-atrum).

Pharmaceutical or veterinary compositions that comprise an antimicrobial effective amount of a CaNCR peptide variant or biologically functional equivalents thereof and a pharmaceutically acceptable carrier are also provided. Such pharmaceutical or veterinary compositions can be used for inhibiting the growth of, or killing, susceptible pathogenic microbes that infect humans or animals, i.e., treating such fungal infections by administering to a patient or other subject in need thereof. In certain embodiments, compositions comprising CaNCR peptide variants and proteins, and biologically functional equivalents thereof, can be formulated by methods such as those described in Remington: The Science and Practice of Pharmacy (2005), 21st Edition, University of the Sciences in Philadelphia, Lippincott Williams & Wilkins. In certain embodiments, the compositions can contain CaNCR peptide variants and proteins, and various combinations thereof, at concentrations in the range of from about 0.1 μg per ml to about 100 mg per ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The CaNCR peptide variants can be formulated alone, in any combination with one another, and either of these can additionally be formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine.

Administration of the compositions that comprise CaNCR peptide variant to a human or animal subject in need thereof can be accomplished via a variety of routes that include topical application.

### Embodiments

The following numbered embodiments form part of the disclosure.

1. A CaNCR peptide variant comprising one or more amino acid substitutions in a CaNCR peptide, wherein said CaNCR peptide comprises a CaNCR13 peptide of SEQ ID NO: 1, a CaNCR7 peptide of SEQ ID NO: 8, a CaNCR14 peptide of SEQ ID NO: 15, or a CaNCR15 peptide of SEQ ID NO: 22, wherein the amino acid substitutions in the CaNCR peptide correspond to a D7A substitution, D7V substitution, D7L substitution, D7I substitution, a D7S substitution, D9A substitution, D9V substitution, a D9L substitution, a D9I substitution, a D9P substitution, a K12W substitution, a K29F substitution, or any combination of said substitutions in a full length reference peptide of SEQ ID NO: 1 when the CaNCR peptide is aligned with SEQ ID NO: 1 at all cysteine residues, and wherein the CaNCR peptide variant has at least 50% sequence identity to SEQ ID NO: 1, 8, 15, or 22.

2. The CaNCR peptide variant of embodiment 1, wherein said CaNCR peptide variant comprises: (i) the amino acid sequence KX₂X₃X₄CX₆X₇X₈KX₁₀, wherein X₂ is K, T, or W, wherein X₃ is F or Y, wherein X₄ is A, R, W, F, or P, wherein X₆ is R or P, wherein X₇ is K, H, T, or P, wherein X₈ is P or S; and wherein X₁₀ is V or W (SEQ ID NO: 29); (ii) the amino acid sequence KWX₃X₄CX₆X₇X₈KW, wherein X₃ is F or Y, wherein X₄ is A, R, W, F, or P, wherein X₆ is R or P, wherein X₇ is K, H, T, or P, and wherein X₈ is P or S (SEQ ID NO: 30); (iii) the amino acid sequence of SEQ ID NO: 6, 13, 20, or 27; or (iv) the amino acid sequence of SEQ ID NO: 7, 14, 21, or 28.

3. The CaNCR peptide variant of embodiment 1 or 2, wherein the CaNCR peptide variant comprises a CaNCR13 peptide variant containing one or more amino acid substitutions in the peptide sequence of SEQ ID NO: 1 selected from the group consisting of a D7A substitution, D7I substitution, a D7S substitution, D9A substitution, a D9I substitution, a D9P substitution, a K12W substitution, a K29F substitution, and any combination of said substitutions, wherein the substitutions are numbered according to the amino acid residue numbering of SEQ ID NO: 1.

4. The CaNCR peptide variant of any one of embodiments 1 to 3, wherein said peptide further comprises an A14W, a V20W, or an A14W and an V20W substitution according to the amino acid residue numbering of SEQ ID NO: 1.

5. The CaNCR peptide variant of embodiment 3, wherein the combination of said substitutions is: (i) a D7I substitution, a D9I substitution, and optionally an A14W, a V20W, or an A14W and an V20W substitution; (ii) a D7S substitution, a D9P substitution, a K12W substitution, and optionally an A14W, a V20W, or an A14W and an V20W; substitution; or a (iii) a D7S substitution, a D9P substitution, a K12W substitution, aK29F substitution, and optionally an A14W, a V20W, or an A14W and an V20W substitution.

6. The CaNCR peptide variant of any one of embodiments 3, 4, or 5, wherein said peptide variant comprises an FACRKPK (SEQ ID NO: 6), KWFACRKPKW (SEQ ID NO: 7), or KWFWCRKPKW (SEQ ID NO: 38) sequence motif.

7. The CaNCR peptide variant of any one of embodiments 3, 4, 5, or 6, wherein said peptide comprises an amino acid sequence having at least 50%, 55%, 60%, 70%, 80%, 84%, 87%, 90%, 93%, or 96% sequence identity across the entire length of SEQ ID NO: 1, optionally wherein said peptide comprises an amino acid sequence having at least 50%, 55%, 60%, 70%, 80%, 84%, 87%, 90%, or 93% sequence identity across the entire length of SEQ ID NO: 1 that lacks the N-terminal threonine (T1) or optionally lacks the N-terminal threonine (T1) and lysine (K2) residues of SEQ ID NO: 1.

8. The CaNCR peptide variant of embodiment 3, wherein the peptide comprises the amino acid sequence of SEQ ID NO: 2, 3, 4, 5, or an N-terminal truncation thereof lacking the N-terminal threonine (T1) or the N-terminal threonine (T1) and lysine (K2) residues of SEQ ID NO: 2, 3, 4, or 5.

9. The CaNCR peptide variant of embodiment 1 or 2, comprising a CaNCR7 peptide variant containing one or more amino acid substitutions in the peptide sequence of SEQ ID NO: 8 selected from the group consisting of a D10I substitution, D10A substitution, D10V substitution, D10L substitution, a D10P substitution, a T13W substitution, a K30F substitution, or any combination of said substitutions, wherein the substitutions are numbered according to the amino acid residue numbering of SEQ ID NO: 8.

10. The CaNCR peptide variant of embodiment 9, wherein said peptide further comprises a P15W, a V21W, or a P15W and aV21W substitution according to the amino acid residue numbering of SEQ ID NO: 8.

11. The CaNCR peptide variant of embodiment 9 or 10, wherein the combination of said substitutions is: (i) a D10P substitution, a T13W substitution, and optionally a V21W substitution; or (ii) a D10P substitution, a T13W substitution, a K30F substitution, and optionally a P15W, a V21W, or a P15W and aV21W substitution.

12. The CaNCR peptide variant of any one of embodiments 9, 10, or 11, wherein said peptide variant comprises YPCPHPK (SEQ ID NO: 13), KWYPCPPSKW (SEQ ID NO: 14), or KWYWCPPSKW (SEQ ID NO: 39) sequence motif.

13. The CaNCR peptide variant of any one of embodiments 9, 10, 11, or 12, wherein said peptide comprises an amino acid sequence having at least 50%, 55%, 60%, 70%, 80%, 84%, 87%, 90%, 93%, or 96% sequence identity across the entire length of SEQ ID NO: 8, optionally wherein said peptide comprises an amino acid sequence having at least 50%, 55%, 60%, 70%, 80%, 84%, 87%, 90%, or 93% sequence identity across the entire length of SEQ ID NO: 8 that lacks the N-terminal lysine (K1) or optionally the N-terminal lysine (K1) and lysine (K2) residues of SEQ ID NO: 8.

14. The CaNCR peptide variant of embodiment 9, wherein the peptide comprises the amino acid sequence of SEQ ID NO: 9, 10, 11, 12, or an N-terminal truncation thereof lacking the N-terminal lysine (K1) or the N-terminal lysine (K1) and lysine (K2) residues of SEQ ID NO: 9, 10, 11, or 12.

15. The CaNCR peptide variant of embodiment 1 or 2, comprising a CaNCR14 peptide variant containing one or more amino acid substitutions in the peptide sequence of SEQ ID NO: 15 selected from the group consisting of a D3I substitution, D3A substitution, D3V substitution, D3L substitution, a D3P substitution, a T6W substitution, a K23F substitution, or any combination of said substitutions, wherein the substitutions are numbered according to the amino acid residue numbering of SEQ ID NO: 15.

16. The CaNCR peptide variant of embodiment 15, wherein said peptide further comprises a P8W, a V14W, or a P8W and a V14W substitution according to the amino acid residue numbering of SEQ ID NO: 15.

17. The CaNCR peptide variant of embodiment 15 or 16, wherein the combination of said substitutions is: (i) a D3I substitution, a T6W substitution, and optionally a P8W, a V14W, or a P8W and a V14W substitution; (ii) a D3P substitution, a T6W substitution, and optionally a P8W, a V14W, or a P8W and a V14W substitution; or (iii) a D3P substitution, a T6W substitution, a K30F substitution, and optionally a P8W, a V14W, and/or a V21W substitution.

18. The CaNCR peptide variant of any one of embodiments 15, 16, or 17, wherein said peptide variant comprises a YPCPPSK (SEQ ID NO: 20), KWYPCPPSKW (SEQ ID NO: 21), or KWYWCPPSKW (SEQ ID NO: 40) sequence motif.

19. The CaNCR peptide variant of any one of embodiments 15, 16, 17, or 18, wherein said peptide comprises an amino acid sequence having at least 50%, 55%, 60%, 70%, 80%, 84%, 87%, 90%, 93%, or 96% sequence identity across the entire length of SEQ ID NO: 15.

20. The CaNCR peptide variant of embodiment 15, wherein the peptide comprises the amino acid sequence of SEQ ID NO: 16, 17, 18, or 19.

21. The CaNCR peptide variant of embodiment 1 or 2, comprising a CaNCR15 peptide variant containing one or more amino acid substitutions in the peptide sequence of SEQ ID NO: 22 selected from the group consisting of a T13W substitution, a K30F substitution, or any combination of said substitutions, wherein the substitutions are numbered according to the amino acid residue numbering of SEQ ID NO: 22.

22. The CaNCR peptide variant of embodiment 21, wherein said peptide further comprises a V21W substitution according to the amino acid residue numbering of SEQ ID NO: 22.

23. The CaNCR peptide variant of embodiment 21 or 22, wherein the combination of said substitutions is: (i) a T13W substitution and a V14W substitution; (ii) a T13W substitution, a K30F substitution, and optionally a V21W substitution.

24. The CaNCR peptide variant of any one of embodiments 21, 22, or 23, wherein said peptide variant comprises a YRCPTPK (SEQ ID NO: 27) or KWYRCPTPKW (SEQ ID NO: 28) sequence motif.

25. The CaNCR peptide variant of any one of embodiments 21, 22, 23, or 24, wherein said peptide comprises an amino acid sequence having at least 50%, 55%, 60%, 70%, 80%, 84%, 87%, 90%, 93%, or 96% sequence identity across the entire length of SEQ ID NO: 22, optionally wherein said peptide comprises an amino acid sequence having at least 50%, 55%, 60%, 70%, 80%, 84%, 87%, 90%, or 93% sequence identity across the entire length of SEQ ID NO: 22 that lacks the N-terminal threonine (T1) or optionally lacks the N-terminal threonine (T1) and lysine (K2) residues of SEQ ID NO: 22.

26. The CaNCR peptide variant of embodiment 21, wherein the peptide comprises the amino acid sequence of SEQ ID NO: 23, 24, 25, or 26.

27. The peptide of any one of embodiments 1-26, wherein said peptide can inhibit microbial growth at a concentration of about 3 μM, optionally wherein the microbial growth is growth of a Fusarium sp., Alternaria sp., Verticillium sp., Phytophthora sp., Colletotrichum sp., Aspergillus sp., Botrytis sp., Cercospora sp., Phakopsora sp. Rhizoctonia sp., Sclerotinia sp., Pythium sp., Erysiphae sp., Blumeria sp., Phoma sp., Gaeumannomces sp. Leptosphaeria sp., Puccinia sp., Diplodia sp., Macrophomina sp., Zymoseptoria sp., Exserohilum sp., Cochliobolus sp., or Heminthosporium sp., or optionally wherein microbial growth is growth of an Aspergillus sp., Candida sp., Histoplasma sp., Paracoccidioides sp., Sporothrix sp., Blastomyces sp., Coccidioides sp., Geomyces sp., Trichophyton sp. or Malassezia sp.

28. The peptide of any one of embodiments 1-27, wherein said peptide comprises SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 or further comprises additional amino acid substitutions wherein one or more of the other hydrophobic, basic, and/or acidic amino acid residues of SEQ ID NO: 2, 3, 4, 5, 9, 10, 11, 12, 16, 17, 18, 19, 23, 24, 25, 26, 34, 35, 36, 37, 46, 47, 48, 49, 50, 51, 52, or 53 are conservatively substituted with hydrophobic, basic, and/or acidic amino acid residues, respectively.

29. The peptide of any one of embodiments 1-28, wherein the cysteine residues corresponding to the cysteine residues of SEQ ID NO: 1, 8, 15, or 22 are conserved.

30. A composition comprising the peptide of any one of embodiments 1 to 28, and an agriculturally, pharmaceutically, or veterinary practicably acceptable carrier, diluent, or excipient.

31. The composition of embodiment 30, wherein the peptide is provided at a concentration of about 0.1, 0.5, 1.0, or 5 μg/ml to about 1, 5, 20, 50, or 100 mg/ml for a liquid composition or at a concentration of about 0.1, 0.5, 1.0, or 5 μg/gram to about 1, 5, 20, 50, or 100 mg/gram for a powder or solid composition.

32. A plant part comprising at least a partial coating and/or infiltrate comprising the composition of embodiment 30 or 31 or a processed plant product obtained therefrom comprising the composition.

33. The plant part of embodiment 32, wherein the plant part is a seed, stem, leaf, root, tuber, flower, or fruit, optionally wherein the peptide is present at a microbe inhibitory concentration, optionally wherein said plant part is non-regenerable.

34. A method for preventing or reducing crop damage by a plant pathogenic microbe comprising the step of contacting a plant, a plant seed, or other part of said plant with an effective amount of the composition of embodiment 30 or 31.

35. The method of embodiment 34, wherein the plant pathogenic microbe is a Fusarium sp., Alternaria sp., Verticillium sp., Phytophthora sp., Colletotrichum sp., Aspergillus sp., Botrytis sp., Cercospora sp., Phakopsora sp. Rhizoctonia sp., Sclerotinia sp., Pythium sp., Erysiphae sp., Blumeria sp., Phoma sp., Gaeumannomces sp. Leptosphaeria sp., Puccinia sp., Diplodia sp., Macrophomina sp., Zymoseptoria sp., Exserohilum sp., Cochliobolus sp., or Heminthosporium sp.

36. A medical device comprising the device and the composition embodiment 30 or 31, wherein the device comprises at least one surface that is topically coated and/or impregnated with the composition.

37. The medical device of embodiment 36 wherein said device is a stent, a catheter, a contact lens, a condom, a patch, or a diaphragm.

38. A method for treating, preventing, or inhibiting a microbial or yeast infection in a subject in need thereof comprising administering to said subject an effective amount of the composition of embodiment 30 or 31.

39. The method of embodiment 38, wherein said administration comprises topical, parenteral, and/or intravenous introduction of the composition.

40. The method of embodiment 38, wherein the subject is a human, livestock, poultry, fish, or a companion animal.

41. The method of embodiment 38, wherein the microbial or yeast infection is of a mucosal membrane, eye, skin, or a nail and the composition is applied to the mucosal membrane, eye, skin, or nail.

42. The method of embodiment 38, wherein the microbial infection is by a dermatophyte.

43. The method of embodiment 42, wherein the dermatophyte is selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

44. The method of embodiment 38, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, Coccidioides or Blastomyces species.

45. The method of embodiment 38, wherein the yeast infection is a Candida species or optionally wherein the Candida species is Candida albicans, C. auris, C. glabrata, C parasilosis, C. tropicalis, or C. krusei.

46. A polynucleotide comprising a DNA or RNA molecule encoding the peptide of any one of embodiments 1 to 29.

47. The polynucleotide of embodiment 46, wherein said DNA molecule further comprises a heterologous promoter that is operably linked to said DNA molecule.

48. A cell comprising the polynucleotide of embodiment 46.

49. The cell of embodiment 48, wherein the cell is a plant, yeast, mammalian, or bacterial cell.

50. The cell of embodiment 49, wherein the cell is a plant cell that is non-regenerable.

51. A plant comprising the polynucleotide of embodiment 46.

52. The plant of embodiment 51, wherein said plant or any part thereof contains a plant pathogenic microbe inhibitory concentration of the peptide.

53. The plant of embodiment 52, wherein the plant pathogenic microbe inhibitory concentration of the peptide is at least 0.005, 0.05, 0.5, or 1 (parts per million) PPM in a tissue or part of the plant.

54. The plant of any one of embodiments 51, 52, or 53, wherein the polynucleotide confers to the plant resistance to infection by a plant pathogenic microbe in comparison to a control plant that lacks the polynucleotide.

55. The plant of embodiment 54, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Gaeumannomces sp. Leptosphaeria sp., or Puccinia sp.

56. The plant of any one of embodiments 51, 52, 53, 54, or 55, wherein the plant is a monocot crop plant or a dicot crop plant.

57. The plant of embodiment 56, wherein said dicot crop plant is not a chickpea plant.

58. The plant of embodiment 56, wherein the monocot crop plant is selected from the group consisting of a corn, barley, oat, pearl millet, rice, sorghum, sugarcane, turf grass, and wheat plant.

59. The plant of embodiment 56, wherein the dicot crop plant is selected from the group consisting of alfalfa, a Brassica sp., cotton, cucurbit, potato, strawberry, sugar beet, soybean, and tomato plant.

60. The plant of any one of embodiments 51, 52, 53, 54, 55, 56, 57, 58, or 59, wherein the plant comprises elite germplasm and/or is not a naturally occurring plant.

61. A plant part obtained from the plant of any one of embodiments 51, 52, 53, 54, 55, 56, 57, 58, 59, or 60, wherein the plant part comprises the polynucleotide.

62. The plant part of embodiment 61, wherein the plant part is a seed, stem, leaf, root, tuber, flower, or fruit.

63. A processed plant product of the plant part of embodiment 61, wherein the processed plant product comprises the polynucleotide.

64. The processed plant product of embodiment 63, wherein the processed plant product comprises de-fatted seed meal or non-defatted seed meal and is optionally non-regenerable.

### EXAMPLES

### Example 1. Antimicrobial Activity of the Plant Antimicrobial CaNCR13 Peptide

The CaNCR13 of SEQ ID NO: 1 was tested for antimicrobial activity against human fungal pathogens.

For expression of the CaNCR13 peptide (SEQ ID NO: 1) in Pichia pastoris, synthetic genes encoding the CaNCR13 peptide were obtained from Genscript (Piscataway, NJ) were cloned in the pPICZαA vector in frame with the α-factor secretion signal sequence without the Glu-Ala repeats at the Kex2 signal cleavage site as described in U.S. patent application Ser. No. 17/309,920 and WO 2020/146360, both incorporated herein by reference in their entireties. The CaNCR13 peptide (SEQ ID NO: 1) from transformed Pichia pastoris were purified to apparent homogeneity (i.e., a single peak) by FPLC and HPLC. The antifungal activity of each peptide was determined against human fungal pathogens as noted in Table 2. The in vitro antifungal activity of these peptides was determined spectrophotometrically as described in previous publications (Sagaram et al. 2013: Islam et al. 2017) in half-strength potato dextrose broth. The MIC values for antifungal activity of each peptide against these pathogens are shown in Table 2.

TABLE 2
In vitro antifungal activity of Pichia pastoris-expressed
CaNCR13 (SEQ ID NO: 1) against human fungal pathogens.

| Pathogen | MIC (μM) |
|---|---|
| Candida albicans (33795) | 3.2 |
| Candida auris (38883) | 6.4 |
| Candida glabrata (38827) | 0.41 |
| Tricophyton rubrum (36381) | 3.2 |
| Tricophyton metagrophytes (26103) | 0.41 |

The CaNCR13 peptide (SEQ ID NO: 1) exhibited antifungal activity at micromolar concentrations against the pathogens used in this study.

### Example 2. Biological Sequences and Associated SEQ ID NO

The amino acid sequences exemplified in the disclosure are described below in Table 3.

TABLE 3
Biological sequences

| SEQ ID | |
|---|---|
| NO. | DESCRIPTION |
| 1 | CaNCR13 wild-type |
| 2 | CaNCR13_V1 (CaNCR peptide variant) |
| 3 | CaNCR13_V2 (CaNCR peptide variant) |
| 4 | CaNCR13_V3 (CaNCR peptide variant) |
| 5 | CaNCR13_v4 (CaNCR peptide variant) |
| 6 | small CaNCR13 core sequence |
| 7 | expanded CaNCR13 peptide variant core sequence, where X2 is |
| | W and X10 is W |
| 8 | CaNCR07 wild type |
| 9 | CaNCR7_V1 (CaNCR peptide variant) |
| 10 | CaNCR7_V2 (CaNCR peptide variant) |
| 11 | CaNCR7_V3 (CaNCR peptide variant) |
| 12 | CaNCR7_V4 (CaNCR peptide variant) |
| 13 | small CaNCR7 core (CaNCR peptide variant) |
| 14 | expanded CaNCR7 peptide variant core sequence, where X2 is |
| | W and X10 is W |
| 15 | CaNCR14 wild type |
| 16 | CaNCR14_V1 (CaNCR peptide variant) |
| 17 | CaNCR14_V2 (CaNCR peptide variant) |
| 18 | CaNCR14_V3 (CaNCR peptide variant) |
| 19 | CaNCR14_V4 (CaNCR peptide variant) |
| 20 | small CaNCR14 core (CaNCR peptide variant) |
| 21 | expanded CaNCR14 peptide variant core sequence, where X2 is |
| | W and X10 is W |
| 22 | CaNCR15 wild type |
| 23 | CaNCR15_V1 (CaNCR peptide variant) |
| 24 | CaNCR15_V2 (CaNCR peptide variant) |
| 25 | CaNCR15_V3, N-terminal truncation of 2 amino acids |
| 26 | CaNCR15_V4, N-terminal truncation of 2 amino acids |
| 27 | small CaNCR15 core |
| 28 | expanded CaNCR15 peptide variant core sequence, where X2 is |
| | W and X10 is W |
| 29 | CaNCR7.13.14.15 Core consensus (KX2X3X4CX6X7X8KX10, |
| | wherein X2 is K, T, or W, wherein X3 is F or Y, wherein X4 is |
| | A, R, W, F, or P, wherein X6 is R or P, wherein X7 is K, H, T, |
| | or P, wherein X8 is P or S; and wherein X10 is V or W.) |
| 30 | CaNCR7.13.14.15 peptide variant core consensus |
| | KWX₃X₄CX₆X₇X₈KW, |
| | wherein X₃ is F or Y, wherein X₄ is A, R, W, F, or P, wherein |
| | X₆ is R or P, wherein X₇ is K, H, T, or P, and wherein X₈ is P |
| | or S |
| 31 | (Gly4)n |
| 32 | (Gly4Ser)n |
| 33 | Ser(Gly4Ser)n |
| 34 | CaNCR13_V5 |
| 35 | CaNCR13_V6 |
| 36 | CaNCR13_V7 |
| 37 | CaNCR13_V8 |
| 38 | Core CaNCR13 substitutions |
| 39 | Core CaNCR7 substitutions |
| 40 | Core CaNCR14 substitutions |
| 41 | NCR13_AlaV1 |
| 42 | NCR13 AlaV2 |
| 43 | NCR13_AlaV3 |
| 44 | NCR13_AlaV4 |
| 45 | NCR13_AlaV5 |
| 46 | CaNCR13_V9 |
| 47 | CaNCR13_V10 |
| 48 | CaNCR13_V11 |
| 49 | CaNCR13_V12 |
| 50 | CaNCR13_V13 |
| 51 | CaNCR13_V14 |
| 52 | CaNCR13_V15 |
| 53 | CaNCR13_V16 |

### Example 3. Alanine Scanning Mutagenesis of the NCR13 Peptide

The chemically synthesized NCR13 and the variants NCR13_AlaV1-V5 (SEQ ID NO: 31-35; Table 3) were obtained from Biomatik Inc, Canada. Each peptide was purified to homogeneity using a linear gradient of acetonitrile/water mixture in a C-18 reverse phase HPLC (Agilent Technologies, CA, USA), with a C18 column. HPLC fractions containing each peptide were lyophilized and resuspended in nuclease-free water. The concentration of each peptide was determined using the BCA assay performed according to the manufacturer's protocol (Thermo-Fisher Scientific). The purity and size of each peptide were verified by sodium dodecyl sulfate-polyacrylamide gel electrophoresis (SDS-PAGE). Mass spectrometric analysis was performed at the Proteomics and Mass Spectrometry Facility at the Donald Danforth Plant Science Center to confirm the correct mass for each peptide.

Antifungal activity of NCR13_AlaV1-V5 was assessed at different concentrations using a 2-fold dilution series of each peptide. Antifungal activity of each peptide was determined spectrophotometrically using the 96-well plate assay. Around 45 μL of each peptide at different concentrations was added to each well of the microtiter plate containing 45 μL of ˜105 spores/ml spore suspension. The quantitative fungal growth inhibition was determined by measuring the absorbance at 595 nm using a (Tecan Infinite M200 ProTecan Systems Inc., San Jose, CA) microplate reader after 48 h. The fungal cell viability was determined by the resazurin cell viability assay. After incubation of the pathogen/peptide mixture for 48 h, 10 μl of 0.1% resazurin solution was added to each well. After incubation of the mixture overnight, a change in the color of the resazurin dye from blue to pink or colorless indicated the presence of live fungal cells. The MIC for each peptide was the lowest concentration of each peptide at which no change in blue color occurred. The results are summarized below in Table 4.

TABLE 4
Antifungal activity of NCR13 and NCR13_AlaV1-V5
was determined against Botrytis cinerea and
Fusarium graminearum

| | | MIC | MIC |
|---|---|---|---|
| | | (μM) | (μM) |
| | | Botrytis | Fusarium |
| Peptide | Sequence (SEQ ID NO) | cinerea | graminearum |
| NCR13 | TKPCQSDKDCKKFACRKPKVPKCINGFC | 3 | 6 |
| | KCVR (SEQ ID NO: 1) | | |
| NCR13_AlaV1 | TKPCAAAAACKKFACRKPKVPKCINGFC | 6 | 6 |
| | KCVR (SEQ ID NO: 41) | | |
| NCR13_AlaV2 | TKPCQSDKDCAAAACRKPKVPKCINGFC | Inactive | Inactive |
| | KCVR (SEQ ID NO: 42) | | |
| NCR13_AlaV3 | TKPCQSDKDCKKFACAAAAVPKCINGFC | Inactive | Inactive |
| | KCVR (SEQ ID NO: 43) | | |
| NCR13_AlaV4 | TKPCQSDKDCKKFACRKPKAAACINGFC | Inactive | 12 |
| | KCVR (SEQ ID NO: 44) | | |
| NCR13_AlaV5 | TKPCQSDKDCKKFACRKPKVPKCAAAAC | 12 | 6 |
| | KCVR (SEQ ID NO: 45) | | |

In vitro antifungal activity of NCR13 and its alanine scanning variants (NCR13_V1-V5) revealed that the sequence motif KKFACRKPKV (SEQ ID NO: 48) is the major sequence motif responsible for the antifungal activity of this peptide (Table 4). The sequence motif VPK also makes a significant contribution to the antifungal activity of NCR13 against B. cinerea and F. graminearum. The sequence motif QSDKD and INGF also make minor contributions to the antifungal activity of this peptide against NCR13 (Table 4).

The breadth and scope of the present disclosure should not be limited by any of the above-described examples.

What is claimed is:

1. A CaNCR peptide variant comprising one or more amino acid substitutions in a CaNCR peptide, wherein said CaNCR peptide comprises a CaNCR13 peptide of SEQ ID NO: 1, a CaNCR7 peptide of SEQ ID NO: 8, a CaNCR14 peptide of SEQ ID NO: 15, or a CaNCR15 peptide of SEQ ID NO: 22, wherein the amino acid substitutions in the CaNCR peptide correspond to a D7A substitution, D7V substitution, a D7L substitution, D7I substitution, a D7S substitution, a D9A substitution, a D9V substitution, a D9L substitution, a D9I substitution, a D9P substitution, a K12W substitution, a K29F substitution, or any combination of said substitutions in a full length reference peptide of SEQ ID NO: 1 when the CaNCR peptide is aligned with SEQ ID NO: 1 at all cysteine residues, and wherein the CaNCR peptide variant has at least 50% sequence identity to SEQ ID NO: 1, 8, 15, or 22.

2. The CaNCR peptide variant of claim 1, wherein said CaNCR peptide variant comprises: (i) the amino acid sequence KX₂X₃X₄CX₆X₇X₈KX₁₀, wherein X₂ is K, T, or W, wherein X₃ is F or Y, wherein X₄ is A, R, W, F, or P, wherein X₆ is R or P, wherein X₇ is K, H, T, or P, wherein X₈ is P or S; and wherein X₁₀ is V or W (SEQ ID NO: 29); (ii) the amino acid sequence KWX₃X₄CX₆X₇X₈KW, wherein X₃ is F or Y, wherein X₄ is A, R, W, F, or P, wherein X₆ is R or P, wherein X₇ is K, H, T, or P, and wherein X₈ is P or S (SEQ ID NO: 30); (iii) the amino acid sequence of SEQ ID NO: 6, 13, 20, or 27; or (iv) the amino acid sequence of SEQ ID NO: 7, 14, 21, or 28.

3. The CaNCR peptide variant of claim 1, wherein the CaNCR peptide variant comprises a CaNCR13 peptide variant containing one or more amino acid substitutions in the peptide sequence of SEQ ID NO: 1 selected from the group consisting of a D7A substitution, D7V substitution, a D7L substitution, D7I substitution, a D7S substitution, a D9A substitution, a D9V substitution, a D9L substitution, a D9I substitution, a D9P substitution, a K12W substitution, a K29F substitution, and any combination of said substitutions, wherein the substitutions are numbered according to the amino acid residue numbering of SEQ ID NO: 1.

4. The CaNCR peptide variant of claim 3, wherein said peptide further comprises an A14W, a V20W, or an A14W and an V20W substitution according to the amino acid residue numbering of SEQ ID NO: 1.

5. The CaNCR peptide variant of claim 1, wherein the combination of said substitutions is: (i) a D7I substitution, a D9I substitution, and optionally an A14W, a V20W, or an A14W and an V20W substitution; (ii) a D7S substitution, a D9P substitution, a K12W substitution, and optionally an A14W, a V20W substitution, or an A14W and an V20W substitution; or a (iii) a D7S substitution, a D9P substitution, a K12W substitution, aK29F substitution, and optionally a an A14W, V20W, or an A14W and an V20W substitution;

6. The CaNCR peptide variant of claim 1, wherein said peptide variant comprises an FACRKPK (SEQ ID NO: 6), KWFACRKPKW (SEQ ID NO: 7), or KWFWCRKPKW (SEQ ID NO: 38) sequence motif.

7. The CaNCR peptide variant of claim 3, wherein said peptide comprises an amino acid sequence having at least 50%, 55%, 60%, 70%, 80%, 84%, 87%, 90%, 93%, or 96% sequence identity across the entire length of SEQ ID NO: 1, optionally wherein said peptide comprises an amino acid sequence having at least 50%, 55%, 60%, 70%, 80%, 84%, 87%, 90%, or 93% sequence identity across the entire length of SEQ ID NO: 1 that lacks the N-terminal threonine (T1) or optionally lacks the N-terminal threonine (T1) and lysine (K2) residues of SEQ ID NO: 1.

8. The CaNCR peptide variant of claim 3, wherein the peptide comprises the amino acid sequence of SEQ ID NO: 2, 3, 4, 5, 34, 35, 36, or 37 or an N-terminal truncation thereof lacking the N-terminal threonine (T1) or the N-terminal threonine (T1) and lysine (K2) residues of SEQ ID NO: 2, 3, 4, or 5.

9. The CaNCR peptide variant of claim 1, comprising a CaNCR7 peptide variant containing one or more amino acid substitutions in the peptide sequence of SEQ ID NO: 8 selected from the group consisting of a D10I substitution, a D10A substitution, a D10V substitution, a D10L substitution, a D10P substitution, a T13W substitution, a K30F substitution, or any combination of said substitutions, wherein the substitutions are numbered according to the amino acid residue numbering of SEQ ID NO: 8.

10. The CaNCR peptide variant of claim 9, wherein said peptide further comprises a P15W, a V21W, or a P15W and a V21W substitution according to the amino acid residue numbering of SEQ ID NO: 8.

11. The CaNCR peptide variant of claim 9, wherein the combination of said substitutions is: (i) a D10P substitution, a T13W substitution, and optionally a P15W, a V21W, or a P15W and a V21W substitution; or (ii) a D10P substitution, a T13W substitution, a K30F substitution, and optionally a P15W, a V21W substitution, or a P15W and a V21W.

12. The CaNCR peptide variant of claim 9, wherein said peptide variant comprises an YPCPHPK (SEQ ID NO: 13) or KWYPCPPSKW (SEQ ID NO: 39) sequence motif.

13. The CaNCR peptide variant of claim 9, wherein said peptide comprises an amino acid sequence having at least 50%, 55%, 60%, 70%, 80%, 84%, 87%, 90%, 93%, or 96% sequence identity across the entire length of SEQ ID NO: 8, optionally wherein said peptide comprises an amino acid sequence having at least 50%, 55%, 60%, 70%, 80%, 84%, 87%, 90%, or 93% sequence identity across the entire length of SEQ ID NO: 8 that lacks the N-terminal lysine (K1) or optionally the N-terminal lysine (K1) and lysine (K2) residues of SEQ ID NO: 8.

14. The CaNCR peptide variant of claim 9, wherein the peptide comprises the amino acid sequence of SEQ ID NO: 9, 10, 11, 12, or an N-terminal truncation thereof lacking the N-terminal lysine (K1) or the N-terminal lysine (K1) and lysine (K2) residues of SEQ ID NO: 9, 10, 11, or 12.

15. The CaNCR peptide variant of claim 1, comprising a CaNCR14 peptide variant containing one or more amino acid substitutions in the peptide sequence of SEQ ID NO: 15 selected from the group consisting of a D3I substitution, D3A substitution, D3L substitution, D3A substitution, a D3P substitution, a T6W substitution, a K23F substitution, or any combination of said substitutions, wherein the substitutions are numbered according to the amino acid residue numbering of SEQ ID NO: 15.

16. The CaNCR peptide variant of claim 15, wherein said peptide further comprises a P8W, a V14W, or a P8W and a V14W substitution according to the amino acid residue numbering of SEQ ID NO: 15.

17. The CaNCR peptide variant of claim 15, wherein the combination of said substitutions is: (i) a D3I substitution, a T6W substitution, and optionally a P8W, a V14W, or a P8W and a V14W substitution; (ii) a D3P substitution, a T6W substitution, and optionally a P8W, a V14W, or a P8W and a V14W substitution; or (iii) a D3P substitution, a T6W substitution, a K30F substitution, and optionally a P8W, a V14W, and/or a V21W substitution.

18. The CaNCR peptide variant of claim 15, wherein said peptide variant comprises a YPCPPSK (SEQ ID NO: 20), KWYPCPPSKW (SEQ ID NO: 21), or KWYWCPPSKW (SEQ ID NO: 40) sequence motif.

19. The CaNCR peptide variant of claim 15, wherein said peptide comprises an amino acid sequence having at least 50%, 55%, 60%, 70%, 80%, 84%, 87%, 90%, 93%, or 96% sequence identity across the entire length of SEQ ID NO: 15.

20. The CaNCR peptide variant of claim 15, wherein the peptide comprises the amino acid sequence of SEQ ID NO: 16, 17, 18, or 19.

21. The CaNCR peptide variant of claim 1, comprising a CaNCR15 peptide variant containing one or more amino acid substitutions in the peptide sequence of SEQ ID NO: 22 selected from the group consisting of a T13W substitution, a K30F substitution, or any combination of said substitutions, wherein the substitutions are numbered according to the amino acid residue numbering of SEQ ID NO: 22.

22. The CaNCR peptide variant of claim 21, wherein said peptide further comprises a V21W substitution according to the amino acid residue numbering of SEQ ID NO: 22.

23. The CaNCR peptide variant of claim 21, wherein the combination of said substitutions is: (i) a T13W substitution and a V14W substitution; (ii) a T13W substitution, a K30F substitution, and optionally a V21 W substitution.

24. The CaNCR peptide variant of claim 21, wherein said peptide variant comprises a YRCPTPK (SEQ ID NO: 27) or KWYRCPTPKW (SEQ ID NO: 28) sequence motif.

25. The CaNCR peptide variant of claim 21, wherein said peptide comprises an amino acid sequence having at least 50%, 55%, 60%, 70%, 80%, 84%, 87%, 90%, 93%, or 96% sequence identity across the entire length of SEQ ID NO: 22, optionally wherein said peptide comprises an amino acid sequence having at least 50%, 55%, 60%, 70%, 80%, 84%, 87%, 90%, or 93% sequence identity across the entire length of SEQ ID NO: 22 that lacks the N-terminal threonine (T1) or optionally lacks the N-terminal threonine (T1) and lysine (K2) residues of SEQ ID NO: 22.

26. The CaNCR peptide variant of claim 21, wherein the peptide comprises the amino acid sequence of SEQ ID NO: 23, 24, 25, or 26.

27. The CaNCR peptide of any one of claims 1 to 26, wherein said peptide can inhibit microbial growth at a concentration of about 3 μM, optionally wherein the microbial growth is growth of a Fusarium sp., Alternaria sp., Verticillium sp., Phytophthora sp., Colletotrichum sp., Aspergillus sp., Botrytis sp., Cercospora sp., Phakopsora sp. Rhizoctonia sp., Sclerotinia sp., Pythium sp., Erysiphae sp., Blumeria sp., Phoma sp., Gaeumannomces sp. Leptosphaeria sp., Puccinia sp., Diplodia sp., Macrophomina sp., Zymoseptoria sp., Exserohilum sp., Cochliobolus sp., or Heminthosporium sp., or optionally wherein microbial growth is growth of an Aspergillus sp., Candida sp., Histoplasma sp., Paracoccidioides sp., Sporothrix sp., Blastomyces sp., Coccidioides sp., Geomyces sp., Trichophyton sp. or Malassezia sp.

28. The CaNCR peptide of any one of claims 1 to 26, wherein said peptide further comprises additional amino acid substitutions wherein one or more of the other hydrophobic, basic, and/or acidic amino acid residues of SEQ ID NO: 2, 3, 4, 5, 9, 10, 11, 12, 16, 17, 18, 19, 23, 24, 25, 26, 34, 35, 36, or 37 are substituted with hydrophobic, basic, and/or acidic amino acid residues, respectively.

29. The CaNCR peptide of any one of claims 1 to 26, wherein the cysteine residues corresponding to the cysteine residues of SEQ ID NO: 1, 8, 15, or 22 are conserved.

30. A composition comprising the peptide of any one of claims 1 to 26, and an agriculturally, pharmaceutically, or veterinarily acceptable carrier, diluent, or excipient.

31. The composition of claim 30, wherein the peptide is provided at a concentration of about 0.1, 0.5, 1.0, or 5 μg/ml to about 1, 5, 20, 50, or 100 mg/ml for a liquid composition or at a concentration of about 0.1, 0.5, 1.0, or 5 μg/gram to about 1, 5, 20, 50, or 100 mg/gram for a powder or solid composition.

32. A plant part comprising at least a partial coating and/or infiltrate comprising the composition of claim 30 or a processed plant product obtained therefrom comprising the composition.

33. The plant part of claim 32, wherein the plant part is a seed, stem, leaf, root, tuber, flower, or fruit, optionally wherein the peptide is present at a microbe inhibitory concentration, optionally wherein said plant part is non-regenerable.

34. A method for preventing or reducing crop damage by a plant pathogenic microbe comprising the step of contacting a plant, a plant seed, or other part of said plant with an effective amount of the composition of claim 30.

35. The method of claim 34, wherein the plant pathogenic microbe is a Fusarium sp., Alternaria sp., Verticillium sp., Phytophthora sp., Colletotrichum sp., Aspergillus sp., Botrytis sp., Cercospora sp., Phakopsora sp. Rhizoctonia sp., Sclerotinia sp., Pythium sp., Erysiphae sp., Blumeria sp., Phoma sp., Gaeumannomces sp. Leptosphaeria sp., Puccinia sp., Diplodia sp., Macrophomina sp., Zymoseptoria sp., Exserohilum sp., Cochliobolus sp., or Heminthosporium sp.

36. A medical device comprising the device and the composition claim 30, wherein the device comprises at least one surface that is topically coated and/or impregnated with the composition.

37. The medical device of claim 36 wherein said device is a stent, a catheter, a contact lens, a condom, a patch, or a diaphragm.

38. A method for treating, preventing, or inhibiting a microbial or yeast infection in a subject in need thereof comprising administering to said subject an effective amount of the composition of claim 30.

39. The method of claim 38, wherein said administration comprises topical, parenteral, and/or intravenous introduction of the composition.

40. The method of claim 38, wherein the subject is a human, livestock, poultry, fish, or a companion animal.

41. The method of claim 38, wherein the microbial or yeast infection is of a mucosal membrane, eye, skin, or a nail and the composition is applied to the mucosal membrane, eye, skin, or nail.

42. The method of claim 38, wherein the microbial infection is by a dermatophyte.

43. The method of claim 42, wherein the dermatophyte is selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

44. The method of claim 38, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, Coccidioides, or Blastomyces species.

45. The method of claim 38, wherein the yeast infection is a Candida species or optionally wherein the Candida species is Candida albicans, C. auris, C. glabrata, C parasilosis, C. tropicalis, or C. krusei.

46. A polynucleotide comprising a DNA or RNA molecule encoding the peptide of any one of claims 1 to 26.

47. The polynucleotide of claim 46, wherein said DNA molecule further comprises a heterologous promoter, which is operably linked to said DNA molecule.

48. A cell comprising the polynucleotide of claim 46.

49. The cell of claim 48, wherein the cell is a plant, yeast, mammalian, or bacterial cell.

50. The cell of claim 49, wherein the cell is a plant cell that is non-regenerable.

51. A plant comprising the polynucleotide of claim 46.

52. The plant of claim 51, wherein said plant or any part thereof contains a plant pathogenic microbe inhibitory concentration of the peptide.

53. The plant of claim 52, wherein the plant pathogenic microbe inhibitory concentration of the peptide is at least 0.005, 0.05, 0.5, or 1 (parts per million) PPM in a tissue or part of the plant.

54. The plant of claim 51, wherein the polynucleotide confers to the plant resistance to infection by a plant pathogenic microbe in comparison to a control plant that lacks the polynucleotide.

55. The plant of claim 54, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Gaeumannomces sp. Leptoshaeria sp., or Puccinia sp.

56. The plant of claim 51, wherein the plant is a monocot crop plant or a dicot crop plant.

57. The plant of claim 56, wherein said dicot crop plant is not a chickpea plant.

58. The plant of claim 56, wherein the monocot crop plant is selected from the group consisting of a corn, barley, oat, pearl millet, rice, sorghum, sugarcane, turf grass, and wheat plant.

59. The plant of claim 56, wherein the dicot crop plant is selected from the group consisting of alfalfa, a Brassica sp., cotton, cucurbit, potato, strawberry, sugar beet, soybean, and tomato plant.

60. The plant of claim 51, wherein the plant comprises elite germplasm and/or is not a naturally occurring plant.

61. A plant part obtained from the plant of claim 51, wherein the plant part comprises the polynucleotide.

62. The plant part of claim 61, wherein the plant part is a seed, stem, leaf, root, tuber, flower, or fruit.

63. A processed plant product of the plant part of claim 61, wherein the processed plant product comprises the polynucleotide.

64. The processed plant product of claim 63, wherein the processed plant product comprises de-fatted seed meal or non-defatted seed meal and is optionally non-regenerable.
